(19) United States
(12) Patent Application Publication
(10) Pub. No.: US 2022/0296522 A1
(43) Pub. Date: Sep. 22, 2022

(54) BRYOID COMPOSITIONS, METHODS OF MAKING AND USE THEREOF

(71) Applicant: Trevor Percival Castor, Arlington, MA (US)

(72) Inventor: Trevor Percival Castor, Arlington, MA (US)

(73) Assignee: Aphios Corporation, Woburn, MA (US)

(21) Appl. No.: 17/359,617

(22) Filed: Jun. 27, 2021

Related U.S. Application Data
(63) Continuation-in-part of application No. 17/207,575, filed on Mar. 19, 2021, now abandoned.

(51) Int. Cl. A61K 9/16; A61K 9/00; A61K 31/366; A61P 25/28

(52) U.S. Cl. CPC A61K 9/1647; A61K 9/0043; A61K 31/366; A61K 9/1682; A61P 25/28

(57) ABSTRACT

Embodiments of the present invention are directed to drug delivery systems, dosage forms and methods for the intranasal administration of Bryostatins for the treatment of neuro-degenerative diseases. Inventions of the present application are directed to the treatment of neuro-degenerative diseases such as Hutchinson Disease, Parkinson's disease, Down syndrome and Alzheimer's disease.

### RELATED APPLICATIONS

This application is a Continuation-in-Part of U.S. patent application Ser. No. 16/208,919, which is a continuation of U.S. patent application Ser. No. 14/647,237, filed May 26, 2015, which is a continuation of 371 U.S. National Phase of International Application No. PCT/US2013/72070, filed Nov. 26, 2013, which claims the benefit of U.S. Provisional Patent Application No. 61/730,227, filed Nov. 27, 2012, the entire contents of which are incorporated herein by reference.

### STATEMENT REGARDING FEDERAL SPONSORSHIP

The inventions of the present application were developed with Federal sponsorship under National Institute of Aging and National Institutes of Health Grant Number 5R44AG034760.

### FIELD OF INVENTION

Embodiments of the present invention are directed to compositions having utility as therapeutics in neurodegenerative diseases such as Hutchinson Disease, Parkinson's disease, Down's syndrome and Alzheimer's disease, virus latency diseases such as HIV and Herpes, cancers such as prostate and other amyloid mediated diseases such as glaucoma.

### BACKGROUND OF THE INVENTION

Neurodegenerative diseases, such as Alzheimer's disease, Hutchinson's Disease, Parkinson's disease, Kuru, Creutzfeldt—Jakob disease and other spongiform encephalopathies remain major health problems. Currently there are very limited means to treat these diseases. With respect to Alzheimer's, Hutchinson's and Parkinson's diseases, these diseases tend to manifest themselves in older individuals and as the diseases progress; the afflicted individuals are less able to care for themselves. The neurogenerative diseases are associated with the formation of beta amyloid plaques.

Bryostatin-1 stimulates the production of certain isoforms of protein kinase C (PKC) that increase the production of alpha-secretase which makes soluble amyloid precursor protein, thus inhibiting the formation of beta amyloid plaques, With respect to cancers such as prostate cancer, Bryostatin-1 inhibits phorbol ester-induced apoptosis in prostate cancer cells by differentially modulating protein kinase C (PKC) delta translocation and preventing PKCdelta-mediated release of tumor necrosis factor-alpha. With respect to virus latency diseases such as HIV latency, Bryostatin-1, as well as many PKC agonists, activates cellular transcription factors such as NF-kB that binds the HIV-1 promoter and regulates its transcriptional activity. In HIV-1 latency the viral promoter is less accessible to cellular transcription factors because nuclear histones surrounding the viral promoter are deacetylated (compacted chromatin). Thus, HDAC inhibitors may increase the acetylation of histones (relaxed chromatin) and then transcription factors may have an easy access to the HIV promoter.

Bryoids consist of a family of bryostatins that are complex cyclic macrolide molecules. Bryoids were originally isolated from the marine bryozoan, Bugula neritina, in small quantities. Methods of synthesis are awkward and costly. About twenty Bryoid compositions, known as bryostatins and numbered 1-20, have been identified. Many of the bryoids are known to possess anti-cancer properties. It would be useful to have new Bryoid compounds that possess high potency and activity.

### SUMMARY OF THE INVENTION

Embodiments of the present invention feature a first Bryoid composition having a molecular weight of approximately 896-898 Amu (Mass+Sodium) having a purity of approximately 50% to a crystal forming purity. The first Bryoid composition can also be characterized as a Bryoid compound having a molecular weight of approximately 873-875 Amu (monoisotopic mass) having a purity of approximately 50% and a crystal forming purity. The first Bryoid composition has a measured mass plus sodium of 897.2 Amu and a measured monoisotopic mass of 874.2 Amu. The detailed discussion which follows will refer to this Bryoid as B10.

Embodiments of the present invention feature a second Bryoid composition having a molecular weight of approximately 910-912 Amu (Mass+Sodium) having a purity of approximately 50% to a crystal forming purity. The second Bryoid composition can also be characterized as a Bryoid compound having a molecular weight of approximately 888-890 Amu (monoisotopic mass) having a purity of approximately 50% and a crystal forming purity. The second Bryoid composition has a measured mass plus sodium of 911.5 Amu and a measured monoisotopic mass of 888.9 Amu. The detailed discussion which follows will refer to this Bryoid as B12.

Embodiments of the present invention feature a third Bryoid composition having a molecular weight of approximately 868-870 Amu (Mass+Sodium) having a purity of approximately 50% to a crystal forming purity. The third Bryoid composition can also be characterized as a Bryoid compound having a molecular weight of approximately 846-848 Amu (monoisotopic mass) having a purity of approximately 50% and a crystal forming purity. The third Bryoid composition has a measured mass plus sodium of 869.5 Amu and a measured monoisotopic mass of 846.6 Amu. The detailed discussion which follows will refer to this Bryoid as B14B.

Embodiments of the present invention feature a fourth Bryoid composition having a molecular weight of approximately 895-897 Amu (Mass+Sodium) having a purity of approximately 50% to a crystal forming purity. The fourth Bryoid composition can also be characterized as a Bryoid compound having a molecular weight of approximately 872-874 Amu (monoisotopic mass) having a purity of approximately 50% and a crystal forming purity. The fourth Bryoid composition has a measured mass plus sodium of 895.5 Amu and a measured monoisotopic mass of 872.6 Amu. The detailed discussion which follows will refer to this Bryoid as B14C.

These Bryoid compounds of the present invention have molecular weights that are different than the molecular weights of bryostatins 1-20.

As used herein, crystal forming purity means the composition has a purity which enables the composition to form crystals. Normally, such purity is greater than 90%, and more often greater than 95% purity. Examples presented in this application feature compositions having a purity greater than 99%. Crystal purity would comprise compositions in which no impurities can be detected, but is not so limited.

The Bryoid composition of the present invention has utility in the treatment of Bryoid responsive conditions such as neurodegenerative diseases, cancers and virus latencies. The Bryoid composition of the present invention is highly active modulators of certain isoforms of protein kinase C (PKC) and amyloid precursor protein. The Bryoids, and the Bryoid composition of the present invention, stimulate the production of certain isoforms of protein kinase C (PKC) that increase the production of alpha (alpha) secretase which transforms amyloid precursor protein into soluble forms. Bryoids composition of the present invention exhibit high levels of activity similar to or greater than bryostatin 1.

One embodiment of the present invention is directed to the treatment of a disease such as a neurodegenerative disease, cancer and virus latency responsive to Bryoids, such as Bryostatins 1-20. The method comprises the step of administering an effective amount of at least one bryoid composition selected from the group consisting of the first Bryoid composition, the second Bryoid composition, the third Bryoid composition, and the fourth Bryoid composition.

Embodiments of the present invention further comprise the Bryoid composition selected from the group consisting of the first Bryoid composition, the second Bryoid composition, the third Bryoid composition, and the fourth Bryoid composition in a dosage form for administration to a patient. The dosage form may take many forms including without limitation, intravenous, intraperitoneal, oral dosage forms, such as tablets, gel caps, capsules, oral solutions and suspensions; aerosols, such as spray or mist forming solutions for administration to lungs, or nasal passageways, topical forms such as ointments, lotions, patches and sprays; and other dosage forms known in the art.

A further embodiment of the present invention is directed to a method of making a Bryoid composition selected from the group consisting of the first Bryoid composition, the second Bryoid composition, the third Bryoid, and the fourth Bryoid composition comprising the steps of isolating a Bryoid composition from a source of Bryoids and purifying the Bryoid composition to a purity of 50% and a crystal forming purity. The source of Bryoids is preferably the marine bryozoan, Bugula neritina.

These and other features and advantages of the present invention will be apparent upon viewing the Figures and reading the detailed descriptions that follow.

### BRIEF DESCRIPTION OF THE FIGURES

FIG. 1 depicts a high-performance liquid chromatography scan of Bryostatin 1.

FIG. 2 depicts a HPLC Chromatogram of B. neritina Ethyl Acetate (EA) crude extract.

FIG. 3 depicts a flow chart of purifying steps for Bryostatin-type compositions.

FIG. 4 depicts alpha-secretase activity induced by Bryostatin-1 and other extracts which embody aspects of the present invention.

FIG. 5 depicts a chromatogram of a mixture of Bryoids.

FIG. 6 depicts a chromatogram of a mixture of Bryoids and identifies retention times monitored at 265 nm.

FIG. 7 depicts UV spectra of different Bryoids at 265 nm.

FIG. 8-1 depicts a mass spectrum of Bryostatin 1, scanning from 700-1000 Amu.

FIG. 8-2 depicts a mass spectrum of a fraction with an internal identification 104 and B08 associated with Bryostatin 2, scanning from 700-1000 Amu.

FIG. 8-3 depicts a mass spectrum of a fraction with internal designations 106 and B14 associated with Bryostatin 3, scanning from 700-1000 Amu.

FIG. 8-4 depicts a mass spectrum of a fraction with internal designations 112 and B16 embodying features of the present invention, scanning from 700-1000 Amu.

FIG. 8-5 depicts a mass spectrum of a fraction with internal designations 102 and B12 and B14 associated with Bryostatin-3, scanning from 700-1000 Amu.

FIG. 8-6 depicts a mass spectrum of a fraction with internal designations 103 and B10 and B12, scanning from 700-1000 Amu.

FIG. 8-7 depicts a mass spectrum of a fraction with internal designations 105 and B12, and B14 associated with Bryostatin-3 scanning from 700-1000 Amu.

FIG. 9 depicts UV spectra of the first Bryoid composition and the second Bryoid composition.

FIG. 10 depicts the effect of Bryostatin-1 and different Bryoids at 10−⁹ M on alpha-secretase activity in SHSY-5Y neuroblastoma cells.

FIG. 11 depicts the effect of Bryostatin-1 and different Bryoids at 10−⁹ M on PKC-epsilon activity in SHSY-5Y neuroblastoma cells.

FIG. 12 depicts the effect of Bryostatin-1 and different Bryoids at 10−⁹ M on PKC-delta activity in SHSY-5Y neuroblastoma cells.

FIG. 13 depicts the effect of Bryostatin-1 and different Bryoids at 10−⁹ M on PKC-alpha activity in SHSY-5Y neuroblastoma cells.

FIG. 14 depicts the proposed structure of a first Bryoid.

FIG. 15 depicts the proposed structure of a second Bryoid.

FIG. 16 depicts the NMR spectra of Bryostatin-3.

FIG. 17 depicts the NMR spectra of the first Bryoid.

FIG. 18 depicts the NMR spectra of the second Bryoid.

FIG. 19 shows a microsphere embodying features of the present invention.

FIG. 20 shows an apparatus for making one or more microspheres of the present invention.

FIG. 21 shows tissue distribution of Bryostatin-1 at 8 h following intranasal administration: Biodistribution of ^{3H}-labeled Bryostatin-1 at 8 h following intranasal administration. Data are normalized to CPM/g. Tracer is most abundant in hippocampus, large intestine and urine.

FIG. 22 shows tissue distribution of Bryostatin-1 at 12 h following intranasal administration: Biodistribution of ^{3H}-labeled Bryostatin-1 at 12 h following intranasal administration. Tracer is most abundant in hippocampus and urine.

FIG. 23 shows tissue distribution of Bryostatin-1 at 24 h following intranasal administration: Biodistribution of ^{3H}-labeled Bryostatin-1 at 24 h following intranasal administration. Tracer is most abundant in hippocampus, urine and feces.

FIG. 24 shows tissue distribution of Bryostatin-1 at 48 h following intranasal administration: Biodistribution of ^{3H}-labeled Bryostatin-1 at 48 h following intranasal administration. Tracer is most abundant in hippocampus and fat tissues. Most tracer now appears in urine/feces.

FIG. 25 shows Hippocampal content of Bryostatin-1 over 72 h following intranasal administration: Hippocampal content of ^{3H}-labeled Bryostatin-1 over 72 h following intranasal administration. Note slow rate of loss over 72 h.

FIG. 26 shows Lung content of Bryostatin-1 over 72 h following intranasal administration: Lung content of ^{3H}-labeled Bryostatin-1 over 72 h following intranasal administration. Note rapid rate of appearance at 4h followed by rapid loss at 8 h.

FIG. 27 shows Improvement in Latency: Bryostatin-1 significantly improves inter-trial latency in a mouse model of Down syndrome. Control differs from Down transgene (TG) (*). Latency improved with 1 μg, (***) and 0.1 μg (*) but not 0.01 μg. *P<0.05, ***P<0.001 vs. WT Error bars shown on SEM.

### DETAILED DESCRIPTION OF THE INVENTION

Embodiments of the present invention will now be described with respect to a Bryoid composition selected from the group consisting of the first Bryoid composition (sometimes referred to as B10), the second Bryoid composition (sometimes referred to as B12), the third Bryoid composition (sometimes referred to as B14B), the fourth Bryoid composition (sometimes referred to as B14C). These Bryoid compounds of the present invention have molecular weights that are different than the molecular weights of Bryostatins 1-20, with the exception of B12 which appears to be a stereoisomer of Bryostatin 3.

Bugula neritina was fractionated to produce Bryostatin fractions (Bryoids) and isolate individual Bryoids.

### HPLC Analysis:

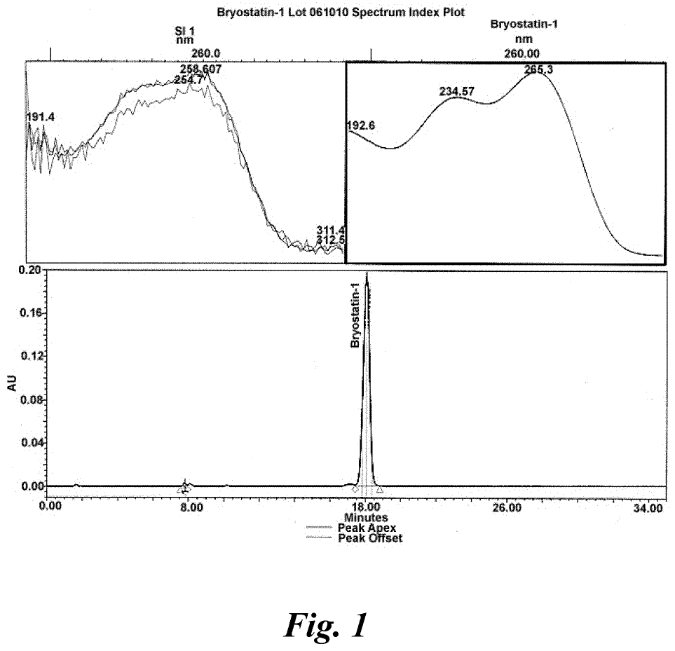

Bryostatin-1 was analyzed by HPLC using a 15 cm 5-micron Phenomenex Luna PFP (2) column (UPS Packing L43) and a mobile phase of 60% acetonitrile acidified with 50 microliters of 85% H₃PO₄ per liter. The flow rate was set to 1.0 mL per minute and the column temperature was set at 30° C. A Waters Millennium system incorporating a Model 996 photodiode array detector was used to generate the chromatographic scans (FIG. 1). Bryostatin-1 was monitored at 265 nm, and contour plots were simultaneously reported from 195 nm to 345 mm

### Bryostatin-1 Manufacturing and Characterization:

In the first two steps, Bryostatins are extracted from wet Bugula neritina with organic solvents including isopropanol, methanol, ethyl acetate and water followed by silica chromatography using mobile phases consisting of hexane/methylene chloride and ethyl acetate/methanol or alternatively extracted from washed, dried and milled Bugula neritina with SuperFluids™ (near-critical and supercritical fluids with or without cosolvents) carbon dioxide and methanol and partially purified by SuperFluids™ silica chromatography with carbon dioxide and methanol (Castor, 1998, 2001).

The third step is a segmentation chromatography step on a CG71 polymeric resin (Rohm-Haas) with a mobile phase consisting of methanol and water that improves the purity of Bryostatin-1 to 60-70%. The fourth step utilizes a segmentation chromatographic method using two semi-prep HPLC C18 columns (Baker Scientific, Phenomenex) with a mobile phase consisting of acetonitrile and water to improve the Bryostatin-1 purity to >95%. The fifth step utilizes crystallization with acetonitrile and water to purify Bryostatin-1 to >98.5%.

The identity of the Bryostatin-1 product was confirmed by Ultra-Violet (UV) spectra as well as High Performance Liquid Chromatography (HPLC) retention times versus those of standards provided by the U.S. National Cancer Institute (NCI), National Institutes of Health (NIH), Bethesda, Md. The identity of the Bryostatin-1 product was also confirmed independently by Mass Spectral (MS) data as well as by Elemental Analysis, Proton and Carbon Nuclear Magnetic Resonance (NMR), Infra-Red (IR) spectroscopy, Differential Scanning calorimetry (DSC) and Melting Point.

### Purification of Bryostatin-1 to 99.64% CP

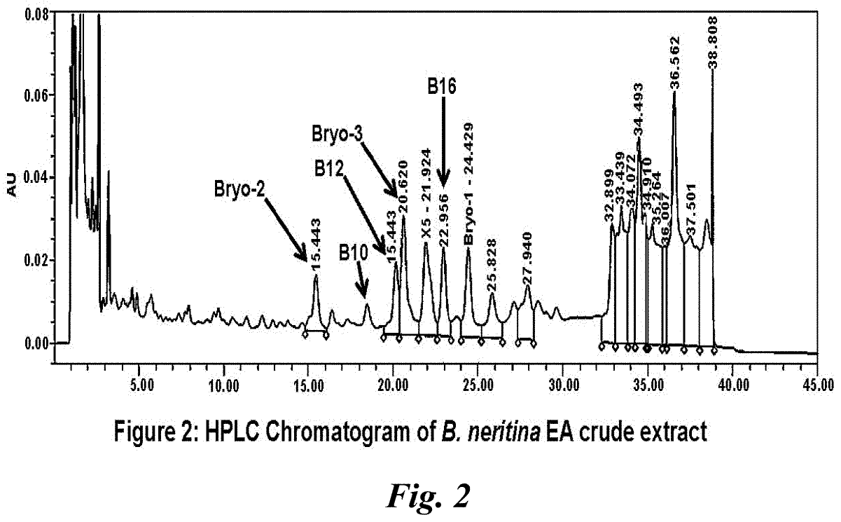

An ethyl acetate extract of B. neritina (Sample C-021519#7), provided by the National Cancer Institute (NCI), and was used as the starting raw materials. A total of ˜57 g of the EA extract was dissolved in dichloromethane (DCM) and assayed to determine presence of Bryostatin-1 and other Bryoids. Turning now to FIG. 2, a HPLC Chromatogram of B. neritina EA crude extract is depicted. Labeled arrows indicate internal designations for Bryostatin-like compounds, which include B10, B12, and Bryostatin 3 (B16), and Bryostatin-2 (Bryo-2) and Bryostatin-3 (Bryo-3). Bryostatin-1 (Bryo-1) elutes at 24.2 min. The designation BIO is the 10ryoid corresponding to the first 10ryoid of the present invention. The designation of B14 will lead to the third and fourth Bryoids of the present invention.

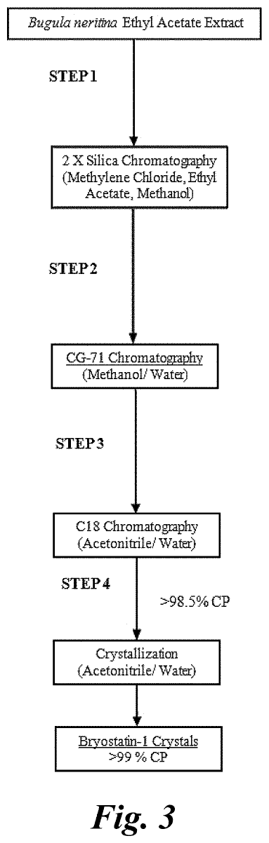

Bryostatin-1 was purified from B. neritina EA crude extracts using various chromatography resins as shown in FIG. 3. The initial steps (Step 1 and Step 2) were performed on Silica gel Active (100-200 μm), and the sample eluted with increasing concentrations of ethyl acetate in DCM. The silica purification steps are useful in removing some of the colored components from the EA crude extract, the non-polar compounds (eluting at end of chromatographic run, FIG. 3), and eliminating the majority of the B16 peak.

Next, fractions containing Bryostatin-1 were purified on Amberchrom CG71, which allowed for the elution of Bryostatin-like compounds with acidified methanol and water. This resin helps minimize the use of chlorinated solvents that are harmful to the environment. CG71 purification step removes the ‘X5’ peak eluting before Bryostatin-1. It also served to minimize the impurities right before Bryostatin-1, mainly B16.

Subsequent purification was performed using a combination of Arnica') C18 40 μm resin and two prep-C18 columns (2.5×2.5 cm, 10 μm column) This step allowed for the further separation of B12 and Bryo-3 from Bryostatin-1, though there was still the presence of the ‘x5’ peak at the shoulder of Bryostatin-1. Final crystallization step led to the purification of Bryostatin-1 to >99% chromatography purity (CP), with a 69% recovery from crude extract.

### HPLC Monitoring:

During each purification step outlined in FIG. 3, Bryostatin-1 was monitored on a Luna C18(2) column (250×4.6 mm, 10 μm). Elution was performed at 80% acetonitrile acidified with phosphoric acid (ACNP) in an isocratic mode at a 2 mL/min flow rate. Column temperature was set at 30° C.

### Bryostatin-Like Compounds (Bryoids)

Bugula neritina was fractionated to produce Bryostatin fractions (Bryoids) that could serve as alternatives to Bryostatin-1. These fractions were purified and sent to LSU for in vitro analysis (Table 1).

TABLE 1
Amount (in mg) of each Bryoid in the
Fractions as determined by HPLC

| | | | | | | Bryo- | | % |
|---|---|---|---|---|---|---|---|---|
| Fraction | Sample | B08 | B10 | B12 | B14 | 1 | B16 | CP* |
| A: 101 | B157 | | | | | 88.8 | | 97.5 |
| | 165 mL | | | | | | | |
| B: 102 | B154 | | | 30.5 | 101.1 | 4.2 | | 74.4 |
| C: 103 | B158 B12 | | 60.4 | 100.8 | 6.7 | | | 49.3 |
| | 350 mL | | | | | | | |
| D: 104 | Bryo-2 | 100.9 | | | | | | 94.4 |
| E: 105 | Bryo-AB | | | 99.8 | 53.7 | | | 64.5 |
| F: 106 | Bryo-3 | | | | 98.3 | | 12.1 | 72.1 |
| G: 112 | B16 APH | | | | | | 99.5 | 97.5 |
| | 100311 | | | | | | | |

*CP corresponds to Bryoid in bold.

Efficacy of Bryostatin-1 Analogues (Bryoids) in Induction of s-APPα Secretion:

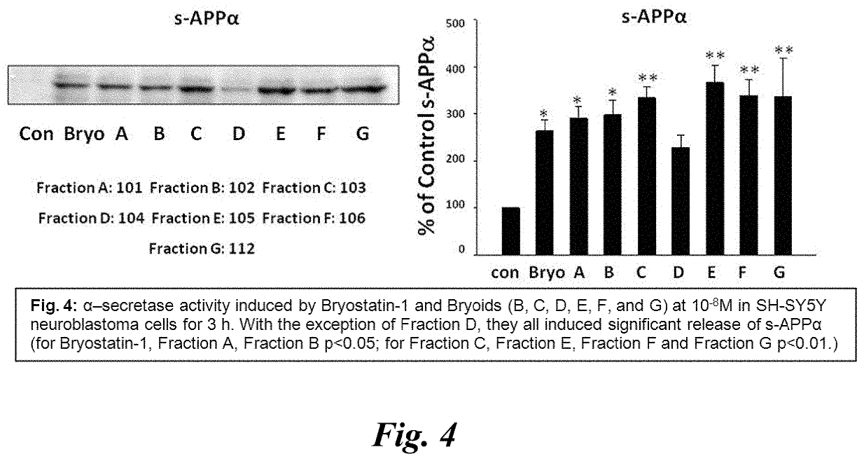

The efficacy of several Bryostatin-1 analogues (Bryoids) in induction of s-APPα secretion is shown in FIG. 4. Except Fraction D, they all induced significant release of s-APPα compared to Bryostatin-1. The best alternative fraction to Bryostatin-1 is analogue E which corresponds to the designation B16, which was identified as Bryostatin 3. Bioactivity went in order from Fraction E (105)>G (112), F (106), C (103)>A (101), B (102)>D (104).

From the preliminary data, it appears that B12 or B14 can be significantly more bioactive than Bryostatin-1. Since most fractions contain two or more Bryoids, it is difficult to determine which one is responsible for the bioactivity except for Fraction G, which contains B16 at >97.5% CP. The first bryoid composition of the present invention, B10, has significantly higher activity than Bryostatin-1, and it poses another potential alternative Bryoid as a therapeutic.

### HPLC Standardization:

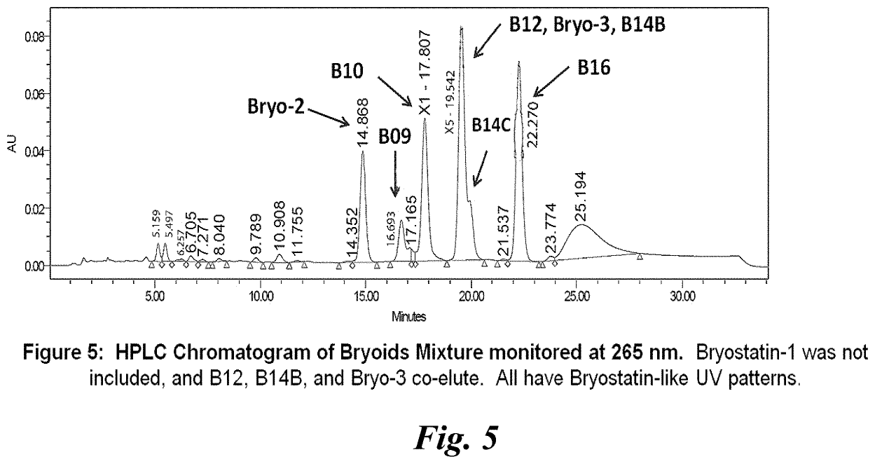

Turning now to FIG. 5, which depicts a high-performance liquid chromatograph of a bryoid mixture at 265 nm, various Bryoids are present in the B. neritina EA crude extract.

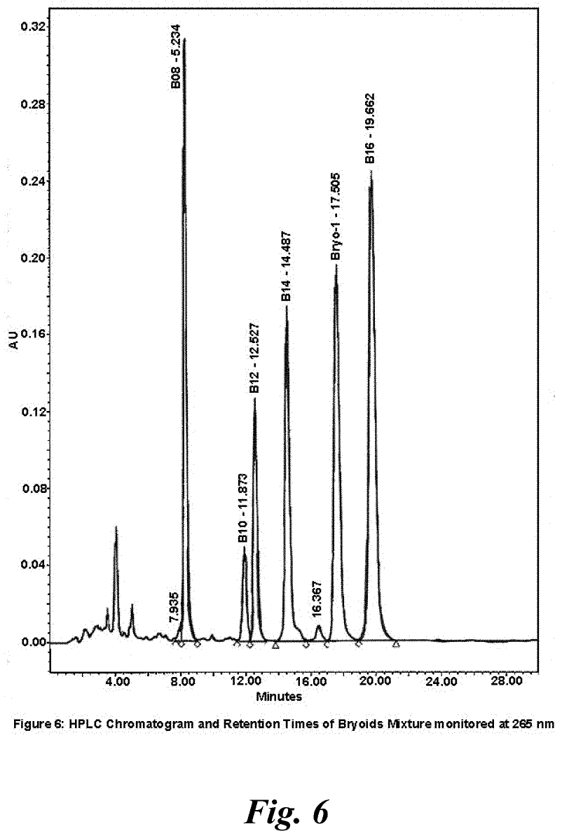

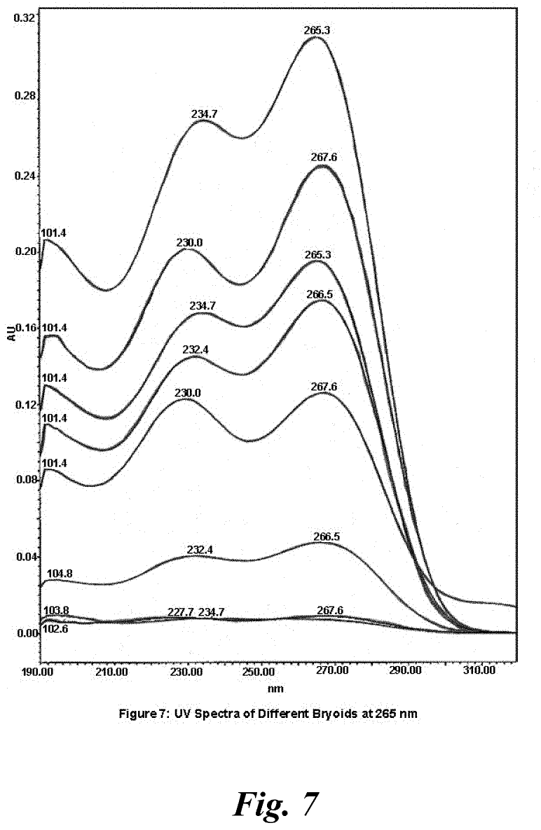

The mixture of the Bryoids (B09, B10, B12, B14C, B14B, B16, Bryostatin-1, Bryostatin-2, and Bryostatin-3) was standardized for the purpose of identification (based on retention time) and subsequent purification of each Bryoid. A chromatogram depicting the results of high-performance liquid chromatography purification is depicted in FIG. 6. These Bryoids contain similar UV patterns as Bryostatin-1 with a maximum wavelength at 265 nm as shown in FIG. 7 which depicts UV spectra of different 13ryoid at 265 mn.

Identification of each Bryoid was attempted on UV-HPLC and LC/MS/MS using known standards and/or comparing to known masses in the literature. This is important as previous preliminary in vitro experiments (described above) have shown that these Bryoids may induce s-APPα secretion at equal or even greater percentages than is observed for Bryostatin-1.

### Preliminary Characterization Based on LC/MS/NIS

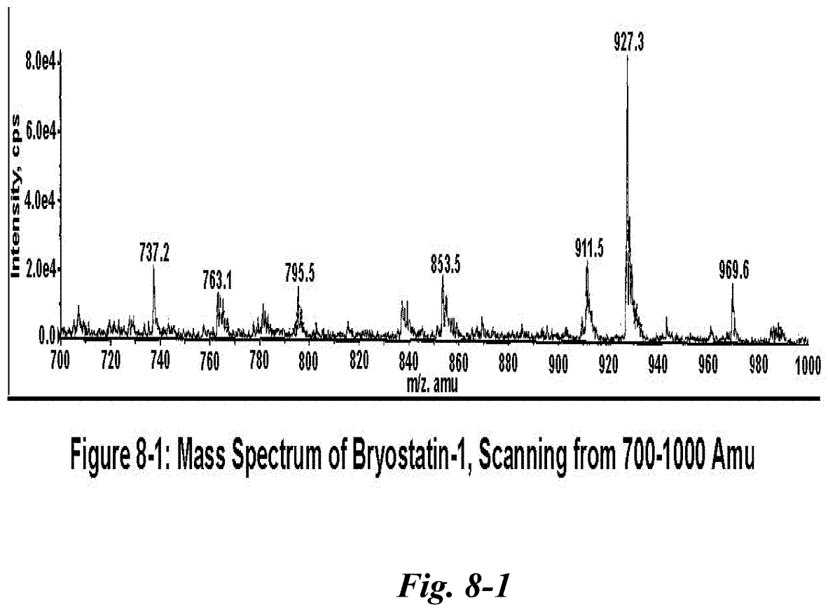

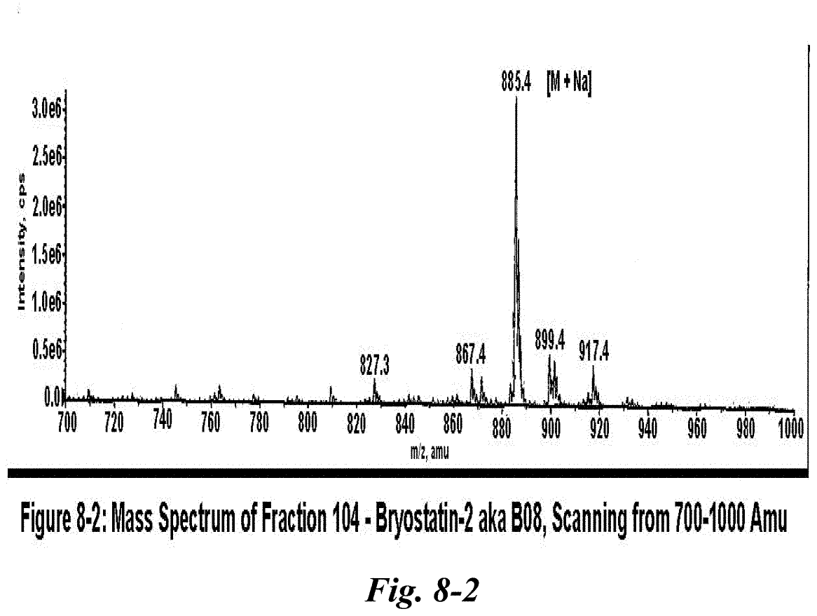

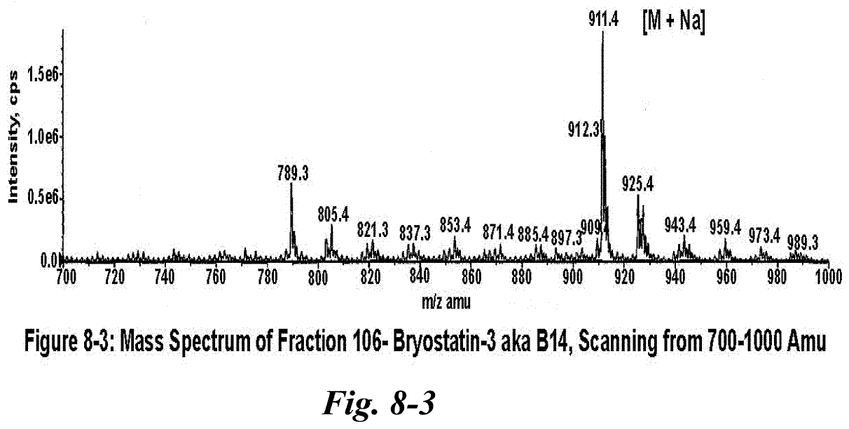

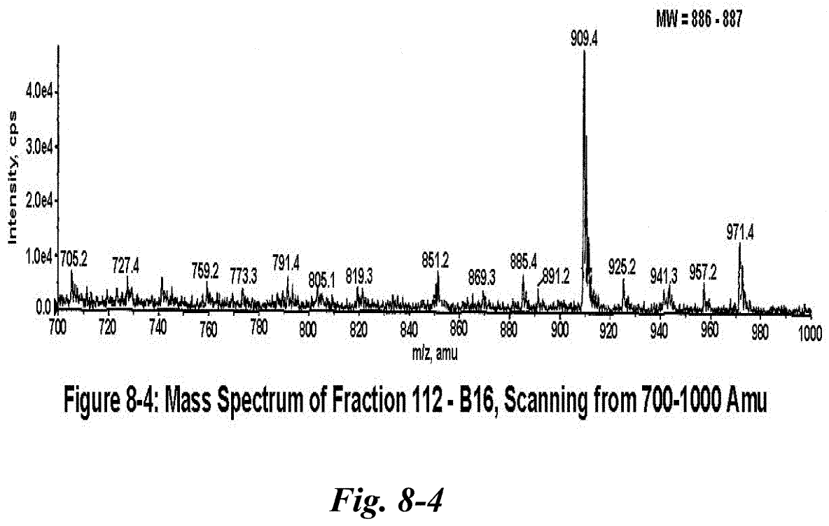

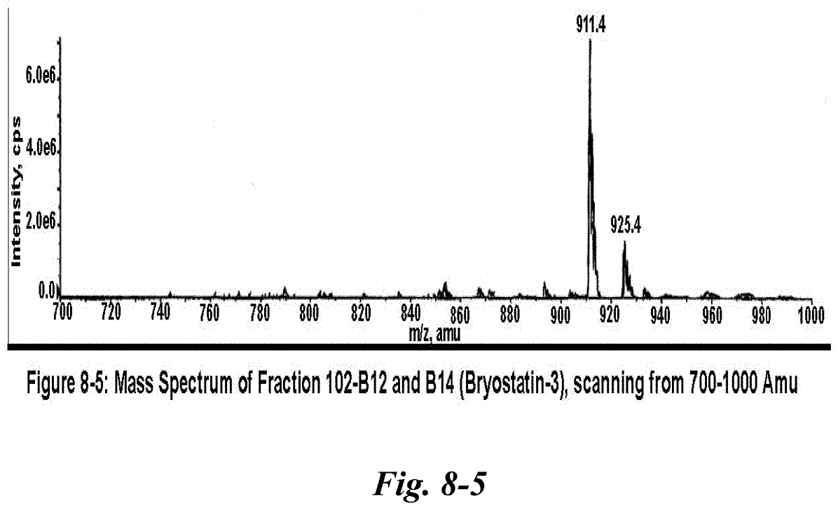

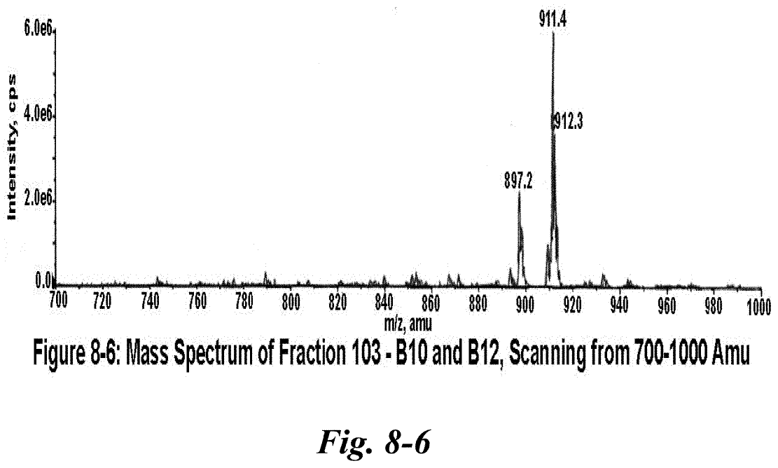

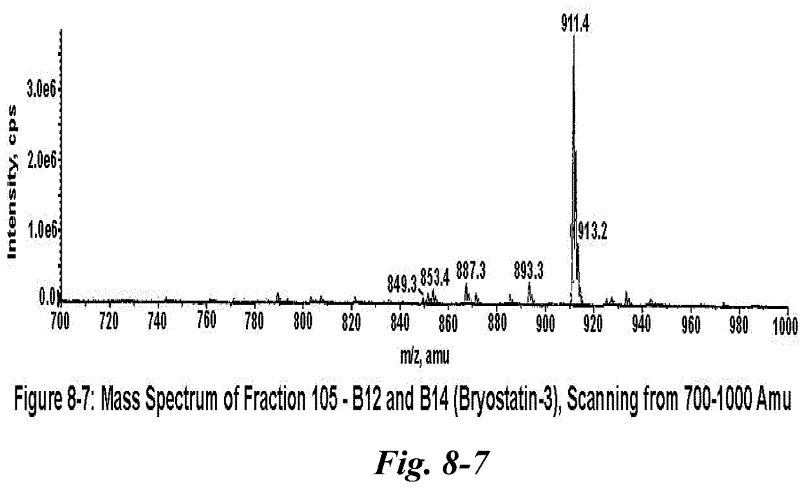

Characterization of the different Bryoids was performed using an LC/MS/MS API 2000 system equipped with a Shimadzu HPLC system. Q1 scan parameters were optimized for Bryostatin-1 m/z 427 [M+Na] (FIG. 8-1), scanning from 700 to 1000 amu. Mass spectrum scans of other fractions are presented in FIGS. 8-2 through 8-7. A total of seven fractions were analyzed, which included individual Bryoids and mixture of Bryoids (Table 2).

TABLE 2
Bryostatin Analogue for Each Fraction Based on Mass Match

| | | | Bryostatin Match |
|---|---|---|---|
| Bryoid | Mass + Na | Mass [M] | Based on Mass |
| Fraction 101: Bryo-1 | 927.3 | 904.3 | Bryostatin-1 |
| Fraction 102. B12 and | 911.4 | 888.4 | Bryostatin-3 |
| B14 (Bryo-3) | 925.4 | 902.4 | None |
| Fraction 103: B10 and | 911.4 | 888.4 | Bryostatin-3 |
| B12 | 897.2 | 874.2 | None |
| Fraction 104: Bryo-2 | 885.4 | 862.4 | Bryostatin-2 |
| Fraction 105: B12 and | 911.4 | 888.4 | Bryostatin-3 |
| Bryo-3 | | | |
| Fraction 106: Bryo-3 | 911.4 | 888.4 | Bryostatin-3 |
| Fraction 112: B16 | 909.4 | 886.4 | None(tentatively |
| | | | identified as |
| | | | Bryostatin-3) |

The LC/MS/MS data observed for Bryostatin-1 shows a peak at 927 Amu, which corresponds to the [M+NA], and what has been reported in the literature (Mannino et al., 2005). Mass spectral data on Bryostatin-1 to 18 are summarized in Table 3. Based on the LC/MS/MS analysis performed, Fractions 104 and Fraction 106 were confirmed as Bryostatin-2 (863 Amu) and Bryostatin-3 (889 Amu), respectively.

Fraction 112 showed that B16 mass does not match any Bryoids reported in the literature. Fractions 102, 103, and 105 showed a mass peak identical to what was observed for Bryostatin-3. Both Fraction 102 and 105 contain Bryo-3 in their mixture, which would explain the 911 peak observed in the LC/MS/MS. It is unclear why 911 Amu is seen in Fraction 103; this indicates that B12 may have the same mass as Bryostatin-3 (889 Amu). This is supported by the fact that Fraction 105, containing both B12 and Bryo-3, only showed a peak at 911 Amu. The 897-peak observed in Fraction 103 could correspond to B10, though it does not match any of the Bryostatin masses reported in the literature. The peak at 925 Amu in Fraction 102 is also observed in Fraction 106.

TABLE 3
Mass Spectral Information on Bryostatin-1 to 18 (Manning et al., 2005)

| | Monoisotopic | M. M. + (Na⁺): | M. M. ± (H₂): | Group R1 monoisotopic | Group R2 monoisotopic | Empirical |
|---|---|---|---|---|---|---|
| Bryo. # | mass | 22.9892 | 2.0156 | mass (attached) | mass (attached) | formula |
| 1 | 904.4456 | 927.4348 | 902.4300 | 59.0133: | 139.0759: | C₄₇H₆₈O₁₇ |
| | | | 906.4613 | CH₃COO | CH₃(CH₂)₂(CH)₄COO | |
| 2 | 862.4350 | 885.4243 | 860.4194 | 17.0027: | 139.0759: | C₄₅H₆₆O₁₆ |
| | | | 864.4507 | OH | CH₃(CH₂)₂(CH)₄COO | |
| 3 | 888.4143 | 911.4035 | 886.3987 | 59.0133: | 139.0759: | C₄₆H₆₄O₁₇ |
| | | | 890.4300 | CH₃COO | CH₃(CH₂)₂(CH)₄COO | |
| 4 | 894.4613 | 917.4505 | 892.4456 | 101.0602: | 87.0446: | C₄₆H₇₀O₁₇ |
| | | | 896.4769 | (CH₃)₂CHCH₂COO | CH₃(CH₂)₂COO | |
| 5 | 866.4300 | 889.4192 | 864.4143 | 101.0602: | 59.0133: | C₄₄H₆₆O₁₇ |
| | | | 868.4456 | (CH₃)₂CHCH₂COO | CH₃COO | |
| 6 | 852.4143 | 875.4035 | 850.3987 | 87.0446: | 59.0133: | C₄₃H₆₄O₁₇ |
| | | | 854.4300 | CH₃(CH₂)₂COO | CH₃COO | |
| 7 | 824.3830 | 847.3722 | 822.3674 | 59.0133: | 59.0133: | C₄₁H₆₀O₁₇ |
| | | | 826.3987 | CH₃COO | CH₃COO | |
| 8 | 880.4456 | 903.4348 | 878.4300 | 87.0446: | 87.0446: | C₄₅H₆₈O₁₇ |
| | | | 882.4613 | CH₃(CH₂)₂COO | CH₃(CH₂)₂COO | |
| 9 | 852.4143 | 875.4035 | 850.3987 | 87.0446: | 59.0133: | C₄₃H₆₄O₁₇ |
| | | | 854.4300 | CH₃(CH₂)₂COO | CH₃COO | |
| 10 | 808.4245 | 831.4137 | 806.4088 | 101.0602: | 1.0078: | C₄₂H₆₄O₁₅ |
| | | | 810.4401 | (CH₃)₃CCOO | H | |
| 11 | 766.3775 | 789.3667 | 764.3619 | 59.0133: | 1.0078: | C₃₉H₅₈O₁₅ |
| | | | 768.3932 | CH₃COO | H | |
| 12 | 932.4769 | 955.4661 | 930.4613 | 87.0446: | 139.0759: | C₄₉H₇₂O₁₇ |
| | | | 934.4926 | CH₃(CH₂)₂COO | CH₃(CH₂)₂(CH)₄COO | |
| 13 | 794.4088 | 817.3980 | 792.3932 | 87.0446: | 1.0078: | C₄₁H₆₂O₁₅ |
| | | | 796.4245 | CH₃(CH₂)₂COO | H | |
| 14 | 824.4194 | 847.4086 | 822.4037 | 101.0602: | 17.0027: | C₄₂H₆₄O₁₆ |
| | | | 826.4350 | (CH₃)₃CCOO | OH | |
| 15 | 920.4405 | 943.4297 | 918.4249 | 59.0133: | 155.0708: | C₄₇H₆₈O₁₈ |
| | | | 922.4562 | CH₃COO | CH₃CH₂CHOH(CH)₄—COO | |
| 16 | 790.4139 | 813.4031 | 788.3983 | 101.0602: | 1.0078: | C₄₂H₆₂O₁₄ |
| | | | 792.4296 | (CH₃)₃CCOO | H | |
| 17 | 790.4139 | 813.4031 | 788.3983 | 101.0602: | 1.0078: | C₄₂H₆₂O₁₄ |
| | | | 792.4296 | (CH₃)₃CCOO | H | |
| 18 | 808.4245 | 831.4137 | 806.4088 | 101.0602: | 1.0078: | C₄₂H₆₄O₁₅ |
| | | | 810.4401 | (CH₃)₃CCOO | H | |

### Isolation of Brvostatin Analogues: B16 (98.5% CP) and B14B (93.4% CP)

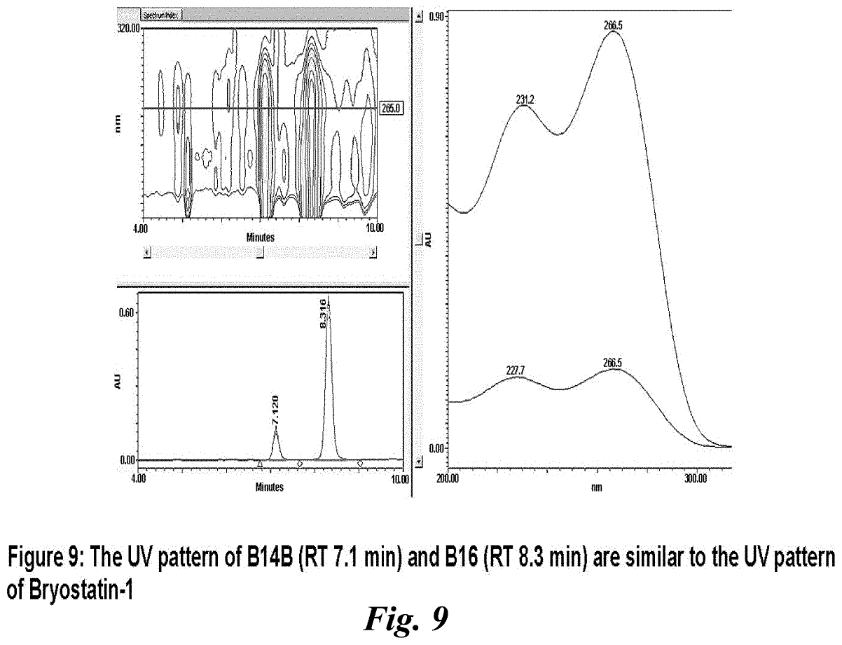

Bryoid-like compounds, B16 and B14B, were purified from side-cuts collected from previous Bryostatin-1 purifications, and had been stored at 4° C. The bryoids' UV-spectra are identical to that of Bryostatin-1 (FIG. 9).

### HPLC Monitoring:

During purification, B16 and B14B were monitored on a Luna CI8 (2) column (250 x 4.6 mm, 10 μm). Elution was performed at 80% ACNP (acetonitrile acidified with phosphoric acid) isocratic mode, at a 2 mL/min flow rate. Column temperature was set at 30° C.

### Purification Procedure and Results:

Fractions containing B16 and B14B were purified using two prep-C18 columns (2.5×2.5 cm, 10 μm) and a semi-prep PEP column. Purification was performed with step-gradient using increasing concentrations of ACNP. Elution was monitored until each Bryoid was located mainly on individual columns. Columns were stripped using a fast gradient with ACNP, and fractions were assayed to determine concentration of each peak.

Bryoids B16 and B14 B can be separated successfully using the described column system. The use of both C18 and PFP column is necessary for the separation of B16 from B14B, and partial purification of B14B from B14C. Peak labeled B14C is another bryoid that co-elutes with B14B, and can be better monitored when analyzed at 70% ACNP. Crystallization of both B16 and B14B/C was possible by addition of MeOH to the Bryoid-containing fractions. A total of 212 mu of B16 crystals with 98.5% CP were collected. A total of 108 mg of B14B/C at 93.4% CP was recovered and stored for future purification. B14B/C was subsequently separated into B14B and B14C. The purified Bryoids were re-analyzed by LC/MS/MS. The results are summarized in Table 4.

TABLE 4
LC/MS/MS Analysis of Purified Bryostatin Analogues
for Each Fraction Based on Mass Match

| | | | Bryostatin Match |
|---|---|---|---|
| Bryoid | Mass + Na | Mass [M] | Based on Mass |
| Bryostatin-1 | 927.3 | 904.3 | Bryostatin-1 |
| Bryostatin-2 | 885.4 | 862.4 | Bryostatin-2 |
| Bryostatin-3 | 911.4 | 888.4 | Bryostatin-3 |
| B16 | 909.4 | 886.4 | None |
| B10 | 897.4 | 874.4 | None |
| B12 | 911.5 | 888.9 | Bryostatin-3 Isomer |
| B14B | 869.5 | 846.6 | None |
| B14C | 895.5 | 872.6 | None |

### Biological Activities of Purified Bryoids:

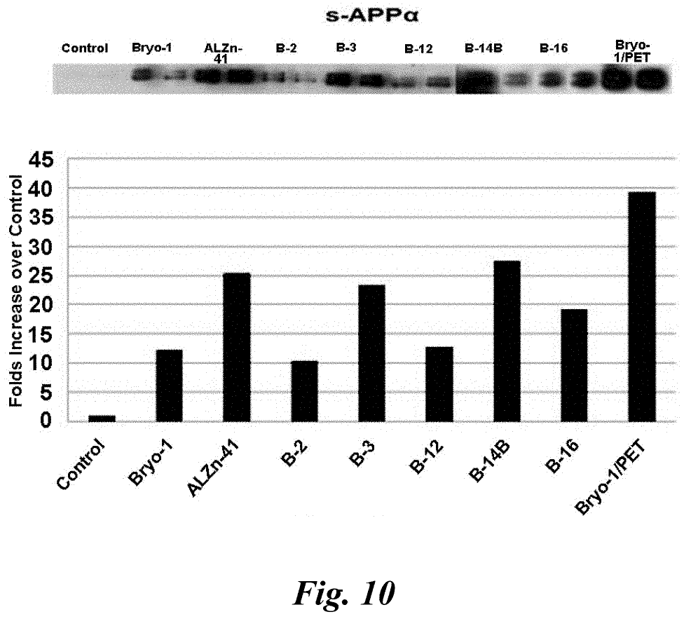

Purified Bryoids at 10−⁹ M are shown to increase alpha-secretase activity in SHSY-5Y neuroblastoma cells in FIG. 10. B3, B14B and B16 are shown to improve the production of alpha-secretase over Bryostatin-1.

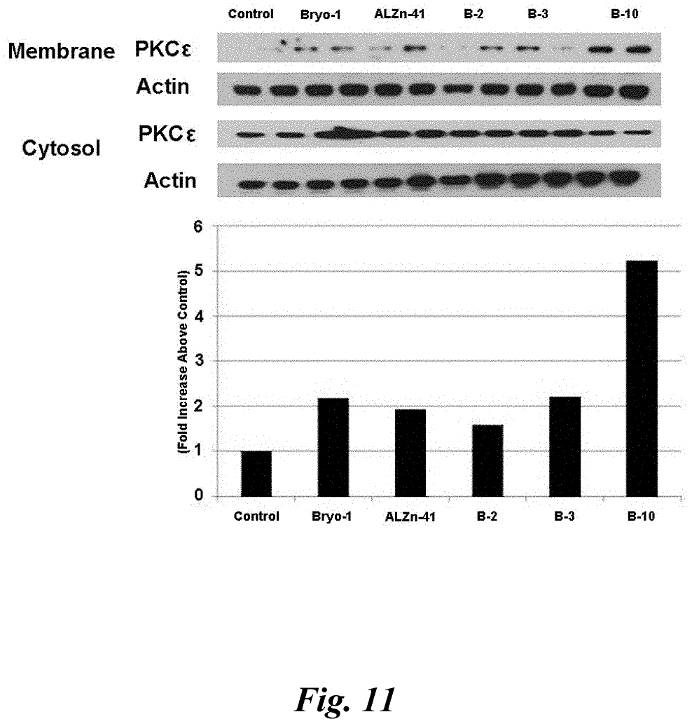

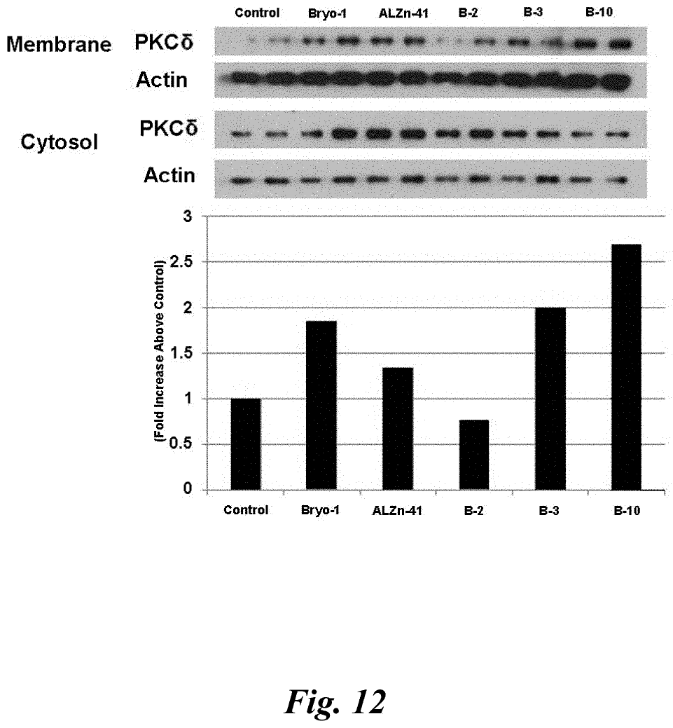

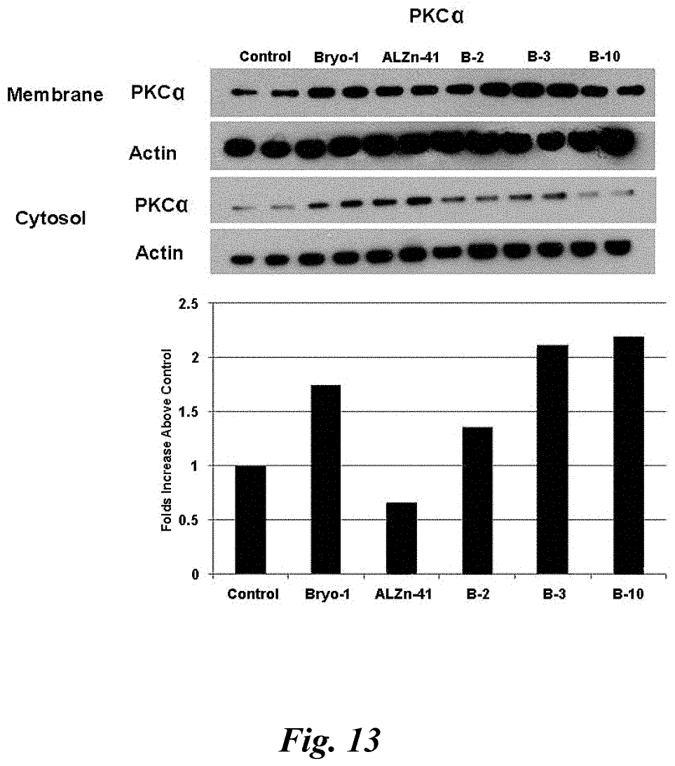

B10 is shown to improve the production of PKC-epsilon over Bryostatin-1 in FIG. 11. B10 is shown to improve the production of PKC-delta over Bryostatin-1 in FIG. 12. B10 is shown to improve the production of PKC-alpha over Bryostatin-1 in FIG. 13.

### NMR and Structural Characterization:

The three variants were compared by to bryostatin 1 and bryostatin 3 by their NMR ¹H and ¹³C resonances and connectivities (HSQC and HMBC spectra). All three variants distinctly had the ring closure at C22 of bryostatin 3, and similar RI and R3 sidechains (the OAc and the 8-carbon 2,4-ene). The variations, relative to bryostatin 3, were:

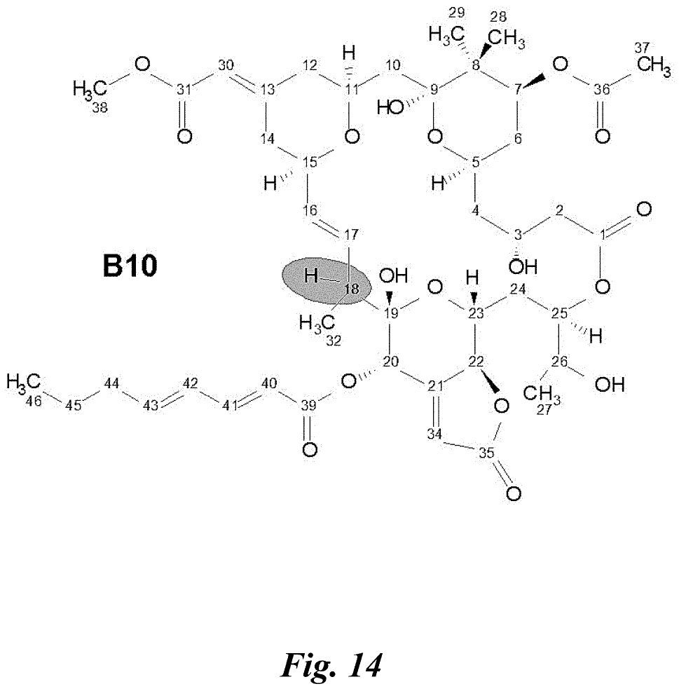

B10: NMR showed loss of one methyl group from C18, matching the mass difference: Predicted C₄₅H₆₂O₁₇=874.4 (monoisotopic); obs B10 874.4. The putative structure of B10 is depicted in FIG. 14.

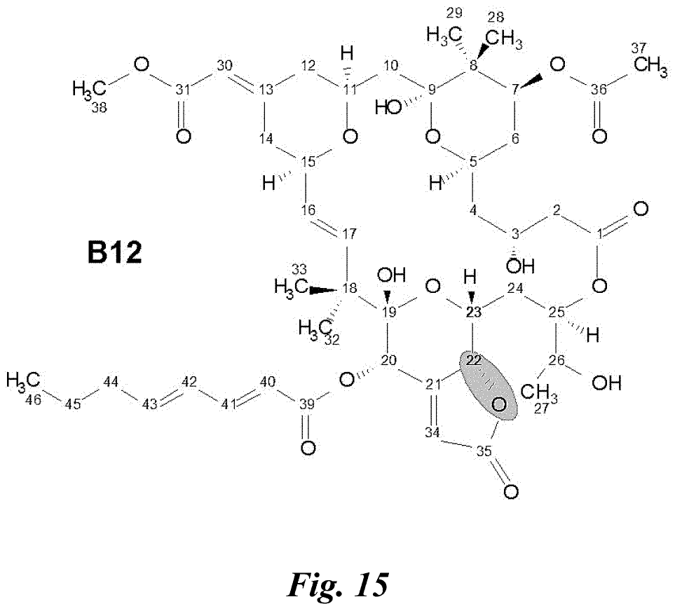

B12 appears to be a stereoisomer: a number of protons in the vicinity of the 19-24 ring have modest changes in chemical shift; but the connectivities show the same covalent structure as bryostatin 3, and it has the same mass as bryostatin 3 (C₄₆H₆₄O₁₇=888.4). The most likely site would be at C22, if the mechanism of ring closure was not perfectly stereoselective. Inversion at adjacent sites (19, 20, or 23) could also explain the NMR changes, although these variations are not seen among the other bryostatins. The putative structure for B12 is depicted in FIG. 15.

In B16, the 26-OH has become a ketone. This change accounts for the 2 Da observed mass difference between B16 (C₄₆H₆₂O₁₇=886.4) and Bryo-3 (888.4). A bryostatin-3 26-ketone is known (Schaufelberger 1991).

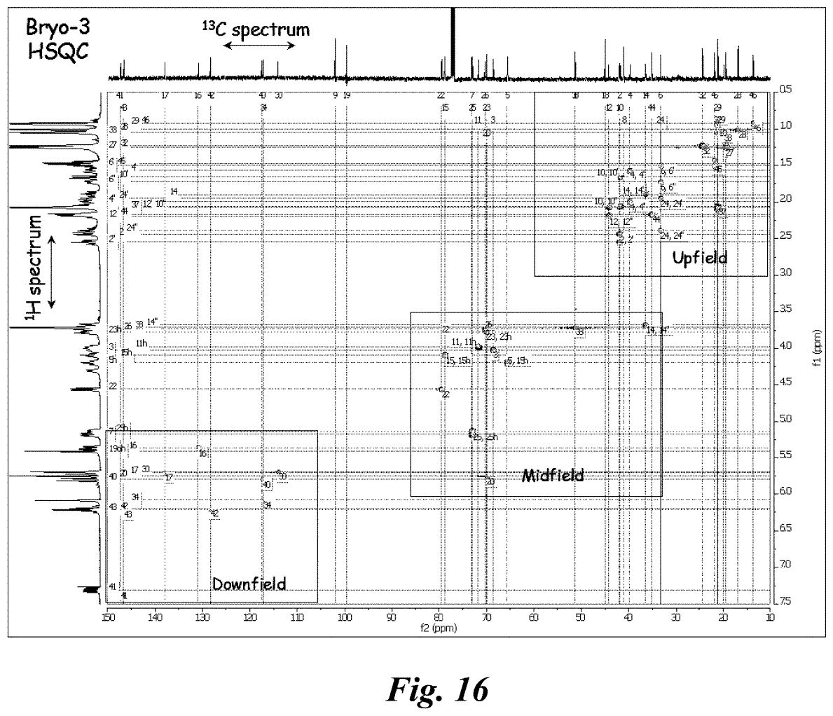

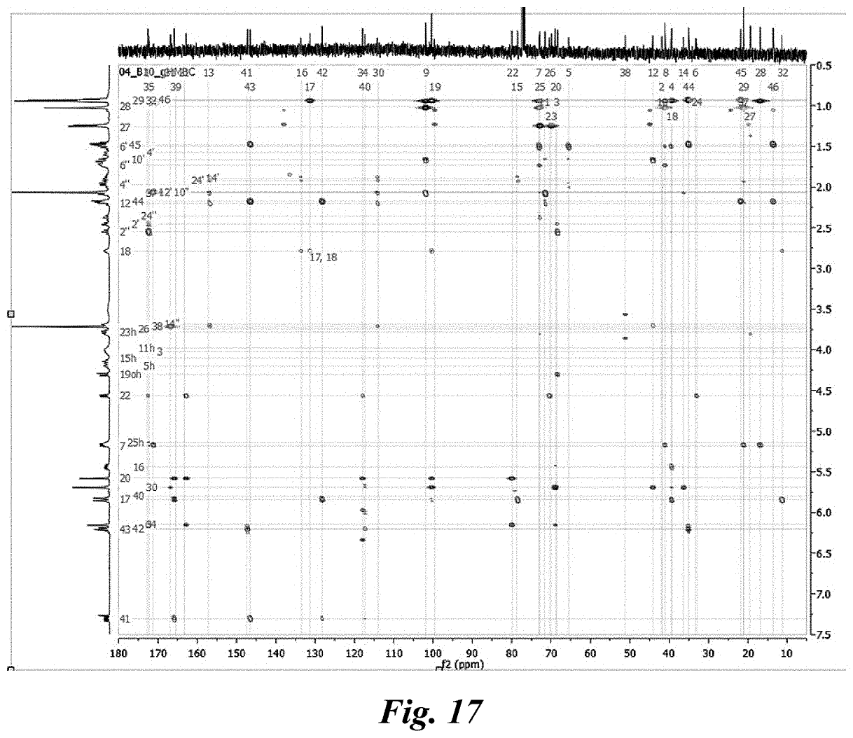

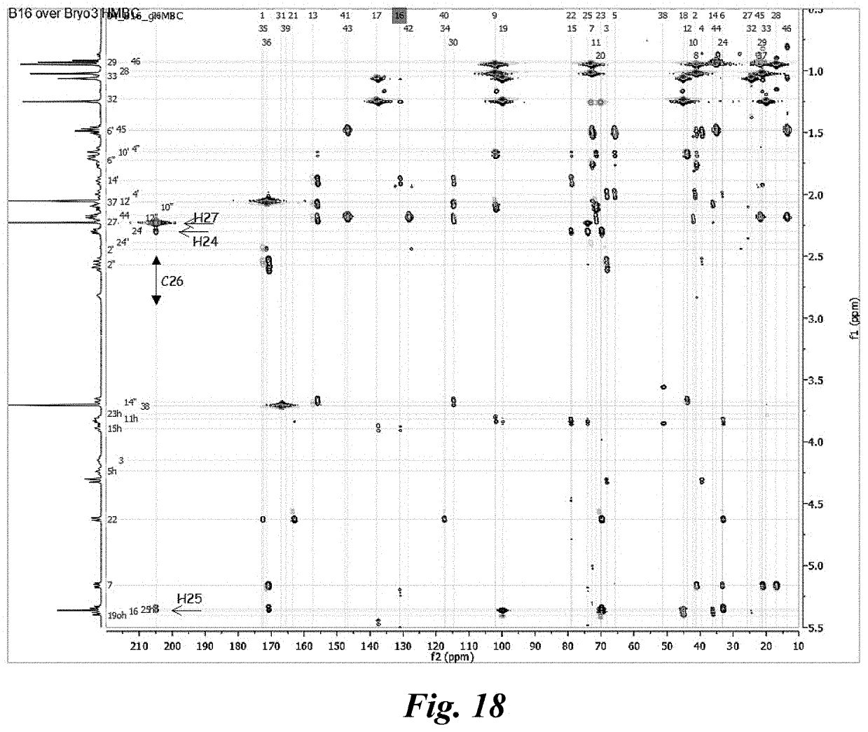

These structures are suggested by the NMR data which is set forth in NMR spectra in FIGS. 16-18. FIG. 16 depicts the NMR spectra of Bryostatin-3. FIG. 17 depicts the NMR spectra of B10. FIG. 18 depicts the NMR spectra of B12 overlaid on the NMR spectra of Bryostatin-3.

### Intranasal and Other Dosage Forms:

Neuro-degenerative diseases, Down syndrome, Parkinson's disease, Kuru, Creutzfeldt-Jakob disease and other spongiform such as Alzheimer's disease, Hutchinson's Disease, and encephalopathies remain major health problems. Currently there are very limited means to treat these diseases. With respect to Alzheimer's, Hutchinson's and Parkinson's diseases, these diseases tend to manifest themselves in older individuals and as the diseases progress; the afflicted individuals are less able to care for themselves. It is therefore highly desirable to have simple therapies which can be administered (e.g. oral and intranasal formulations) without the need for specially trained healthcare providers.

Embodiments of the present invention are directed to drug delivery systems, dosage forms and methods for the treatment of neuro-degenerative diseases. Turning first to embodiments directed to an article of manufacture, one embodiment features an effective amount of a Bryostatin-1 in a biopolymer. The biopolymer comprises a plurality of microspheres in which the spheres have a diameter between one to 1000 nanometers. The neuro-degenerative diseases which are the object of treatment in the present invention are exemplified by Alzheimer's disease, Hutchinson's Disease, Parkinson's disease, Kuru, Creutzfeldt—Jakob disease, Down syndrome and spongiform encephalopathies.

As used herein, the term “a Bryostatin” refers to any and all Bryostatins and derivatives thereof Twenty-two Bryostatins have been identified and certain examples feature a Bryostatin that is Bryostatin-1.

Embodiments of the present invention feature a biopolymer which is resistant to acid. For example, without limitation, one biopolymer is a poly (D, L-lactide-co-glycolic acid). This biopolymer has two components. Embodiments of the present invention feature a poly (D, L-lactide-co-glycolic acid) having a ratio of lactide and glycolic acid of 25-75% lactide with the remaining comprising glycolic acid. A common ratio is 50:50 lactide to glycolic acid as determined by weight. This biopolymer is resistant to gastric acid degradation and allows oral delivery of the drug to the small intestine for absorption.

Embodiments of the present invention feature spheres that are lyophilized for reconstitution in an aqueous solution. Another embodiment features spheres held in suspension for oral administration and/or held in an oral dosage form selected from the group of tablets, capsules, gel caps, and powders. Suspensions for oral administration are preferably flavored to improve patient acceptance.

Another embodiment features spheres held in suspension for intranasal administration and/or held in an intranasal dosage form selected from the group of droplets, mists, sprays and powder.

A further embodiment of the present invention is directed to a method of treating neuro-degenerative disease. The method comprises the steps of administering an effective amount of a Bryostatin held in a plurality of spheres, each sphere comprising a biopolymer and Bryostatin, and each sphere having a diameter of one to 1000 nanometers.

Embodiments of the present method feature a Bryostatin selected from the group consisting of Bryostatins 1-20.

One embodiment of the present invention features a biopolymer which is resistant to acid. For example, without limitation, one acid resistant biopolymer is a poly (D, L-lactide-co-glycolic acid). Poly (D, L-lactide-co-glycolic acid) has a ratio of lactide and glycolic acid. A preferred ratio is 25-75% lactide with the remaining comprising glycolic acid.

Preferably, the microspheres are lyophilized for reconstitution in an aqueous solution, or held in suspension for oral or intranasal administration or held in an oral dosage form selected from the group of tablets, capsules, gel caps, and powders. Intranasal dosage forms include sprays, mists, powders and droplets.

As a further article of manufacture, embodiments of the present invention feature an effective amount of a Bryostatin dissolved in pharmaceutically acceptable oil for oral administration for the treatment of neuro-degenerative disease. As used herein, the term “pharmaceutically acceptable oil” refers to oils which are reasonably well tolerated for oral ingestion in small amounts of 5 to 10 milliliters. Embodiments of the present invention feature olive oil. Other embodiments comprise, by way of example, without limitation include, cotton seed oil, cod liver oil, castor oil, safflower oil, peanut oil, sesame oil, corn oil, vegetable oils, oils originating with animals, and other oils commonly used in the food industry. The oil is preferably administered in a gel cap.

An effective amount of Bryostatin for humans is about 0.1 to 3.0 mg per day in the pharmaceutically acceptable oil and approximately 100 micrograms to 2 mg per day as in the microsphere. Effective dosing for intranasal administration can range from 0.1 μg to 10 μg Bryostatin with preferred range from 0.5 to 2.0 μg.

A further embodiment of the present invention is directed to a method of treating neuro-degenerative disease comprising the steps of administering orally an effective amount of a Bryostatin dissolved in pharmaceutically acceptable oil, or administering intranasally in sprays, mists, or droplets.

Thus, as a treatment for neuro-degenerative diseases, embodiments of the present invention feature dosage forms and methods for the oral and intranasal administration of an effective amount of a Bryostatin. These and other features and advantages of the present invention will be apparent upon reading the text of the detailed description below as well as viewing the accompanying drawings.

Embodiments of the present invention will be described with respect to a drug delivery system, dosage form and method for the treatment of neuro-degenerative diseases exemplified by Alzheimer's disease and Down syndrome, with the understanding that the discussion relates to other neuro-degenerative diseases as well. This discussion will feature the preferred embodiments of the invention with the understanding that features of the invention are capable of modification and alteration without departing from the teaching.

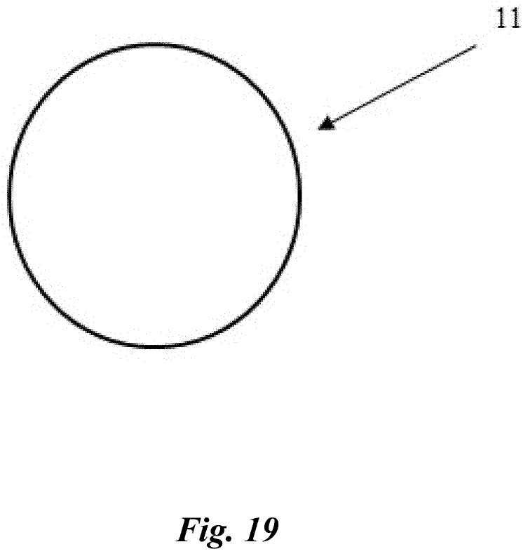

FIG. 19 shows a microsphere, generally designated by the numeral 11 embodying features of the present invention, is depicted. The microsphere 11, when combined with an adequate number of like microspheres comprises an effective dose of a Bryostatin in a biopolymer. Each microsphere 11 has a diameter of one to 1000 nanometers. Although depicted as a microsphere, the article of manufacture may have an irregular shape, roughness, or be filamentous in form.

As used herein, the term “a Bryostatin” refers to any and all Bryostatins and derivatives thereof. Examples of the present invention feature ‘Bryoids’ which is a term that refers to a naturally occurring fractions of Bryostatins purified to about 95% chromatographic purity. Bryostatins are isolated in accordance with Castor, U.S. Pat. No. 5,750,709 and Castor “Supercritical fluid Isolation of Bryostatin-1, Phase II Final Report, SBIR Grant No. 5 R44 CA64017-03, Apr. 21, 2001.

Embodiments of the present invention feature a biopolymer resistant to acid. For the purpose of the present discussion, resistance to acid refers to stomach acids at a pH of approximately 1 to 3 for a period of time of about 0.5 to 4.0 hours. One biopolymer is a poly (D, L-lactide-co-glycolic acid). This biopolymer has two components, a lactide and a glycolic acid component. Embodiments of the present invention feature a poly (D, L-lactide-co-glycolic acid) having a ratio of lactide and glycolic acid of 25-75% lactide with the remaining comprising glycolic acid. A common ratio is 50:50 lactide to glycolic acid as determined by weight. This biopolymer is resistant to acid degradation and allows oral delivery of the drug to the small intestine for absorption.

Embodiments of the present invention feature microspheres that are lyophilized for reconstitution in an aqueous solution. Another embodiment features microspheres held in suspension for oral administration and/or held in an oral dosage form selected from the group of tablets, capsules, gel caps, and powders. Methods of making tablets, capsules, gel caps and powders are well known in the art. (Remington, The Science and Practice of Pharmacy'—20^{th} Edition Lippincott, Williams and Williams). Suspensions for oral administration are preferably flavored to improve patient acceptance.

Another embodiment of the present invention features microspheres held in suspension for intranasal administration in the dosage form of sprays, mists, and droplets.

Another embodiment of the present invention features pharmaceutically orally acceptable oil containing an effective amount of Bryostatin. An amount of oil for administration is determined, and an effective amount of Bryostatin is dissolved in such oil in a manner known in the art. Preferably, the amount of oil which is intended for oral administration is enclosed in a gel cap in a manner known in the art. For example, Vitamin D and Vitamin E supplements are often enclosed in gel cap formulations.

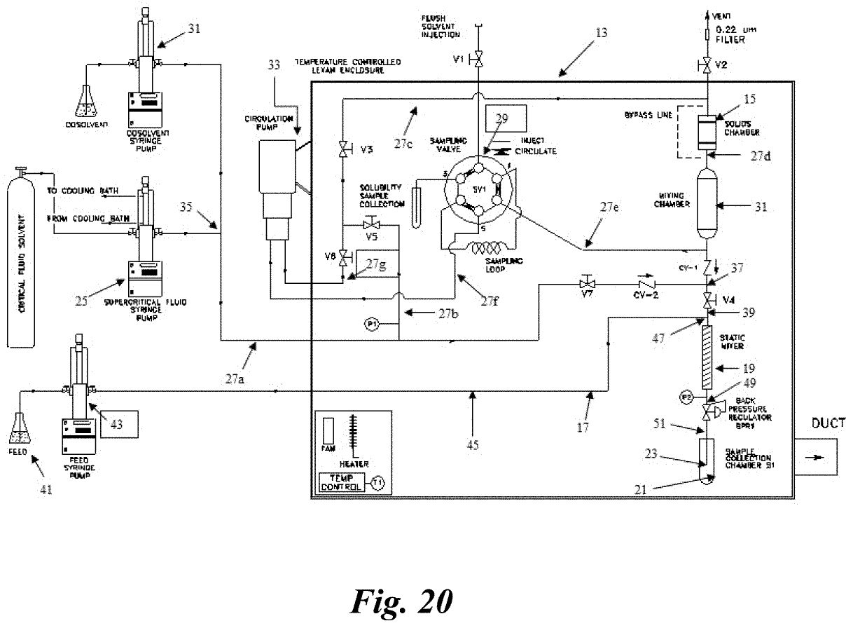

The present method and apparatus will be described with respect to FIG. 20 which depicts in schematic form a polymer sphere apparatus, generally designated by the numeral 13. The polymer sphere apparatus is comprised of the following major elements: a polymer vessel 15, a Bryostatin drug injection assembly 17, an admixture chamber 19, a depressurization vessel 21, and an orifice nozzle 23.

Polymer vessel 15 is in fluid communication with a supercritical critical or near critical syringe pump 25 via conduits 27a, 27b and 27c. Supercritical, critical or near critical pump 25 is in fluid communication with a source of supercritical, critical or near critical fluid.

Polymer vessel 15 is also in fluid communication with a modifier syringe pump 31 via conduit 33 which intersects with conduit 27a at junction 35. Modifier syringe pump 31 is in communication with a source of modifiers and/or entrainers (not shown).

Polymer vessel 15 is loaded with polymer. This polymer vessel receives supercritical, critical or near critical fluid from supercritical critical or near critical pump 25 via conduits 27a, 27b and 27c. Polymer vessel 15 receives modifiers and/or entrainers from modifier pump 31 via conduit 33. Polymer is dissolved in the supercritical, critical or near critical fluid and modifier to form a polymer solution. Formation of the polymer solution is facilitated by circulating the polymers and supercritical, critical or near critical fluid in a loop with a conduits 27d, 27d, 27e, 27f, and 27g, a master valve 29, a mixing chamber 31, and a circulation pump 33.

Polymer vessel 15 is in fluid communication with admixture chamber 19 via conduits 37 and 39. Admixture chamber 19 is also in fluid communication with Bryostatin drug injection assembly 17. Bryostatin drug injection assembly 17 comprises Bryostatin drug syringe pump 43, a source of a Bryostatin 41 and conduit 45. Bryostatin drug syringe pump 43 is in communication with a source of Bryostatin material and pressurizes and compels such material through conduit 45. Conduit 45 is in communication with admixture chamber via conduits 39 which intersects conduit 45 at junction 47. Preferably, junction 47 is a mixing “T”.

Admixture vessel 19 is in the nature of an inline mixer and thoroughly mixes incoming streams from the polymer vessel 15 and Bryostatin drug injection assembly 17. Admixture vessel 19 is in communication with orifice nozzle 23 via conduit 49. Orifice nozzle 23 is in the nature of a back-pressure regulator and has a nozzle defining one or more orifices which discharge into depressurization vessel 21 via conduit 51. Preferably orifice nozzle 23 controls pressure and decompression rates such that a supercritical critical or near critical carbon dioxide enters the orifice at a rate of about 0.425 mL/min and 0.075 mL/min acetone or about 0.5 mL/min carbon dioxide and ethanol combined to maintain system pressure at 2,500 psig.

The operating pressure of the system can be preset at a precise level via a computerized controller (not shown) that is part of the syringe pumps. Temperature control in the system is achieved by enclosing the apparatus 11 in ¼″ Lexan sheet while utilizing a Neslab heating/cooling system coupled with a heat exchanger (not shown) to maintain uniform temperature throughout the system.

In a typical experimental run, polymeric materials were first packed into the polymer vessel 15. Supercritical critical or near critical fluid and an ethanolic solution of Bryostatin drug were charged into the supercritical, critical or near critical syringe pumps 25 and 31, respectively, and brought to the desired operating pressure. An ethanol solution of Bryostatin drug is charged into bioactive syringe pump 43.

The system is pressurized with the supercritical critical or near critical fluid via supercritical, critical or near critical syringe pump 25 to the pressure level equal to that set-in modifier syringe pump 31 and Bryostatin drug syringe pump 43, and maintained at this level with the nozzle orifice 23. The dynamic operating mode for all pumps is set so that each pump can be operated at its own desired flow rate. The supercritical critical or near critical stream flows through the polymer vessel 15, dissolves polymer and contacts the Bryostatin drug stream at junction 47. The mixture of supercritical critical nears critical fluid, Bryostatin drug and polymer materials is then passed through admixture chamber 19 for further mixing. Finally, the mixed solution entered orifice nozzle 23 and was injected into a 10% sucrose solution containing 0.1% polyvinyl alcohol, with or without 40% ethanol with or without trace acetic acid in the depressurization vessel 21. As a result of supercritical fluid decompression, polymer spheres containing Bryostatin drug are formed in the 10% sucrose solution, 0.1% polyvinyl alcohol, with or without 40% ethanol with or without trace acetic acid. The expanded supercritical fluid exits the system via a vent line on the depressurization vessel 21.

The polymer spheres are in the nature of microspheres 11. These microspheres 11 are frozen at −80° degrees Centigrade and lyophilized.

Oil based Bryostatin solutions are dissolved in olive oil with vitamin E as a preservative and lecithin and medium chain triglyceride emulsifiers to increase bioavailability. The oil with the dissolved Bryostatin is encapsulated in gel capsules with a nitrogen purge and head. In the alternative, the oil with dissolved Bryostatin is administered as a liquid dosage form. However, those skilled in the art recognize that oily formulations are not normally well received due to taste and texture. The oil with dissolved Bryostatin may also be emulsified and administered as a liquid formulation. Emulsification may mask some of the less desirable taste and texture associated with oil based oral formulations.

### EXAMPLES

### Bryostatin Microspheres:

Microspheres comprising polymers and Bryostatin 1 were prepared in accordance with the methods described above. The results are summarized in Table 5 below.

TABLE 5
Summary of Polymer Nanoencapsulation of Bryostatin-1 Experiments

| | | P | T | Particle Size | Bryo-1 | Encapsulation |
|---|---|---|---|---|---|---|
| Expt. No. | SFS | (bars) | (° C.) | (nm) | (mg/100 mL) | (%) |
| ALZ-01-01 | CO₂:Acetone::95:5 | 171 | 45 | 259 | 0.0511 | 11.4 |
| ALZ-02-01 | Freon-22 | 205 | 22 | 973 | 0.3089 | 16.8 |
| ALZ-03-01 | CO₂:Ethanol::85:15 | 171 | 45 | 246* | 0.0027 | 71.3 |
| ALZ-04-01 | CO₂:Acetone::95:5 | 171 | 45 | 215* | 0.0160 | 50.8 |
| ALZ-05-01 | CO₂:Acetone::95:5 | 171 | 45 | 254* | 0.1323 | 84.0 |
| ALZ-06-01 | CO₂:Acetone::85:15 | 171 | 45 | 251* | 0.2374 | 82.3 |

*After lyophilization and reconstitution

The nanospheres appear stable at 4-25° C. (Centigrade) for at least one-week duration. Further, the nanospheres appear stable in solutions at about pH 1.13 at 37° C. (Centigrade), similar to a stomach environment.

Results further suggest that nanospheres with Bryostatins and Bryostatin 1, in particular, induce alpha-secretase processing of amyloid precursor protein (APP) to s-APP alpha, and activate protein kinase C (PKC) isoforms alpha, delta and epsilon (measured by membrane translocation) in the SH-SY5Y neuroblastoma cell line. These events are well-described cell and pharmacological events associated with prevention of beta-secretase mediated formation of beta-amyloid, the presumptive cause of dementia in human Alzheimer's disease and in the sweAPP/PS1 mouse model of Alzheimer's disease.

### Oil-Based Formulations for Liquid-Fill Gel Capsules:

Based on the hydrophobicity of Bryostatin-1, we developed an oil-based formulation of Bryostatin-1. A stock solution of 82 mg/100 mL of Bryostatin-1 was used. Isopropyl alcohol, Extra Virgin olive oil, sesame oil, and vegetable oil were all used as solvents. Thirty microliters of the stock solution were placed in each of 4 clean, dry HPLC vials. The ethanol was allowed to evaporate, leaving 25 micrograms in the vial. Then, 1.0 mL of the solvent was placed in the vial and vortexed to ensure proper mixing. These samples were then injected on a normal phase HPLC system, with a gradient of 10%-70% isopropyl alcohol in hexane as the mobile phase (specifically developed for this experiment). The concentration of each vial theoretically should be 2.5 mg/100 mL. The results are listed in Table 6.

TABLE 6
Concentrations of Bryostatin in Different Solvents

| | Concentration |
|---|---|
| Solvent | (mg/100 mL) |
| Isopropyl Alcohol | 2.6035 |
| Extra Virgin Olive Oil | 2.9945 |
| Vegetable Oil | 2.5475 |
| Extra Virgin olive containing mixed natural | 2.4431 |
| tocopherol antioxidants to improve stability, | |
| and lecithin and medium chain triglyceride | |
| emulsifiers to increase bioavailability. | |

The data in Table 6 indicates that Bryostatin-1 is soluble in a variety of different types of oil. The reason for the higher concentrations than the standard (isopropyl alcohol) is due to the baseline. While attempting a baseline subtraction for each oil, there was negative absorbance so the blank IPA sample was subtracted from each sample's baseline. While this incorporates a little more area for integration, the amount of Bryostatin in the oil was quantifiable. In addition, the sesame oil had an integration area that was much larger than the peak itself When manipulating the review application within the Millennium HPLC software, it was seen that the peak itself had a similar area to that of the standard (Bryostatin in IPA).

Bryostatin-1 is soluble in a variety of oils, with the best results in Extra Virgin Olive Oil, Vegetable Oil, and Extra Virgin Olive Oil with excipients. Bryostatin-1 is formulated to a specific concentration in Extra Virgin olive containing mixed natural tocopherol antioxidants to improve stability, and lecithin and medium chain triglyceride emulsifiers to increase bioavailability. This formulation is then encapsulated in gel capsules with a N₂ purge and head. Targeted concentrations are in the range of 10 to 25 μg/mL.

### Water Maze Studies:

Mouse strain B6C3-Tg carrying mutant Swedish Amyloid precursor protein (sweAPP) and PS I (presenilin-1) genes associated with early onset Alzheimer's disease were subjected to water maze tests at 5-6 months of age. These tests suggest that mice that received Bryostatin-1 at a dose of 5 micrograms/mouse on alternative days orally in an oil 20 formulation showed significant protection against Alzheimer's disease mediated memory loss produced by the APP/PS1 mutations as compared with memory acquisition skills seen in control animals.

In Vivo Studies with Bryostatin-1 Formulations:

In vivo studies were conducted using the Morris water maze to evaluate cognitive impairments and restoration in response to drug treatments. These studies used the mouse strain B6C3-Tg (APPswe, PSEN1 dE9) 85Dbo/J mice (MMRRC, Jackson Labs).

In vivo studies were also conducted on the intranasal administration of Bryostatin-1 in the TS65DN transgenic mouse model of Down syndrome because of the genetic similarity of Down syndrome to Alzheimer's disease.

We discovered that Bryostatin-1 improves inter-trial latency in a mouse model of Down's syndrome. We found that compared to wild type mice, the TS65DN model exhibited significantly poorer task acquisition, especially on day 3. In this model, wild type mice improved over the 4 trials shown as a reduction in latency within the trial. We also found that mice which had been treated with 1 μg Bryostatin-1 showed a significant improvement in inter-trial water maze performance, with a p<0.001 compared to vehicle treated transgenic Down's mice. Interestingly, mice which were treated with 0.1 μg Bryostatin-1 also showed an inter-trial interval improvement (p<0.05) compared to vehicle treated Down's mice, but mice treated with 0.01 μg did not show this same improvement. This shows a significant difference in task acquisition in the Down's syndrome model which shares many characteristics with Alzheimer's disease. Importantly, these data show for the first time a dose dependent improvement in task performance with 1 and 0.1 μg Bryostatin-1, while 0.01 μg did not.

These are also the first data to show that intranasal Bryostatin-1 can affect performance in the TS65DN model which is important because our radioactive uptake data have demonstrated high levels of Bryostatin-1 uptake into the hippocampus. This approach indicates that intranasal delivery of Bryostatin-1 may represent an important and novel treatment modality in human Down syndrome.

We also conducted extensive in vivo pharmacokinetic and pharmacodynamics studies with radio-labeled Bryostatin-1 to determine metabolism, excretion, bioavailability and biodistribution by different administration routes, oral (gavage), intra-peritoneal (i.p.), intravenous (i.v.) with a Z-oil formulation, and intranasal (i.n.) with a PET formulation.

We found that the accumulation of radiolabeled Bryostatin-1 in different tissues was lowest by the oral route with the highest tissue accumulations in the i.p. and i.v. dosed groups. By comparison, intranasal delivery of Bryostatin-1 appeared to achieve relative high levels of Bryostatin-1 accumulation in the hippocampus and lung by 4 h. Although i.p. treatment appeared to produce good biodistribution we did not find that i.p. treated mice showed as good responses in the water maze studies. This suggests that the mode of delivery and to a lesser extent the overall amount delivered may influence ‘efficacy’ in this model. This in vitro data suggests that Bryostatin-1 is not significantly metabolized by SK-HEP1 liver cells within 24 h.

The efficacy of Bryostatin-1 nanospheres and Bryostatin-1 in PET formulation to induce s-APPα secretion in SH-SYSY neuroblastoma cells were evaluated in vitro. The PET formulation was designed primarily for intravenous administration, was later evaluated for intranasal pharmacokinetics in normal mice and efficacy in a transgenic mouse model of AD.

In vitro studies with brain endothelial and neuron cell cultures reveal mechanisms for the trans BBB exchange of Bryostatin-1, although our recent introduction of intranasal delivery of Bryostatin-1 may overcome this physical barrier to allow lower dosing schedules with enhanced effectiveness at lower delivered doses.

We have now evaluated the differences in uptake of radiolabeled Bryostatin-1 depending on the route of administration using 4 different routes of administration: 1) oral (oil formulation), 2) intravenous, 3) intraperitoneal (oil formulation) and 4) intranasal (PET formulation). It is worth mentioning that oil formulations were used for oil and intraperitoneal studies. Aqueous formulations were used for intravenous administration and PET formulation for intranasal administration.

### Intranasal Delivery of Brvostatin-1:

Intranasal delivery may accomplish better delivery to the hippocampus, the anticipated target of Bryostatin-1 in memory cognition studies, than oral, intravenous, and intraperitoneal administration. Intranasal administration may be in the form of sprays, mists, powders and droplets, and dosing can range from 0.1 μg to 10 μg with preferred range from 0.5 to 2.0 μg.

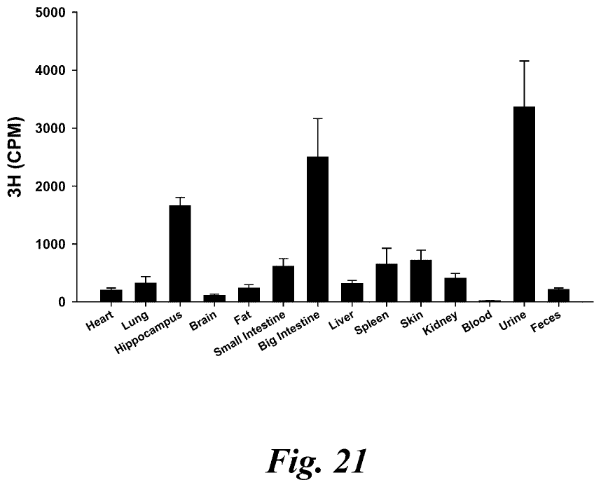

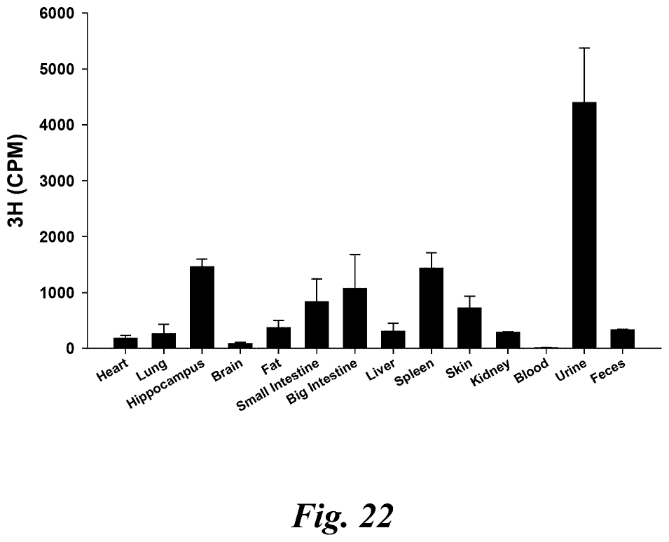

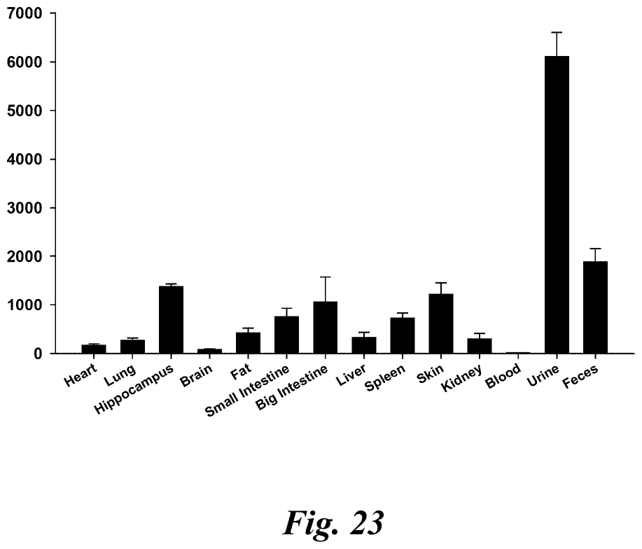

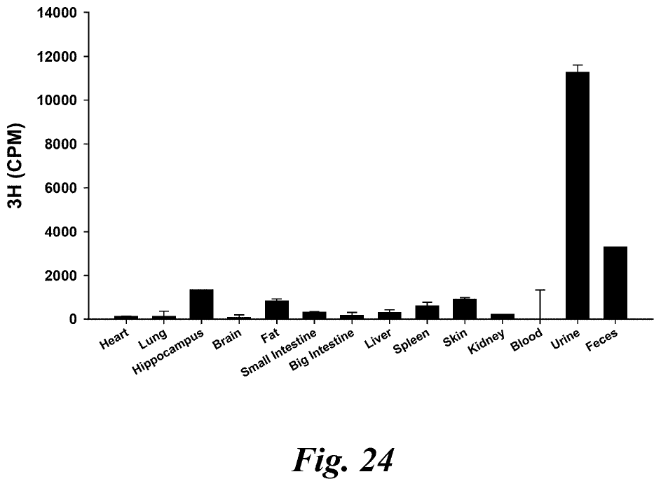

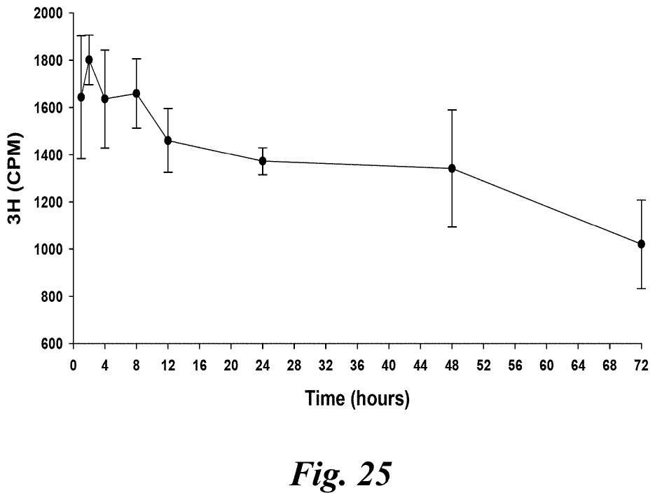

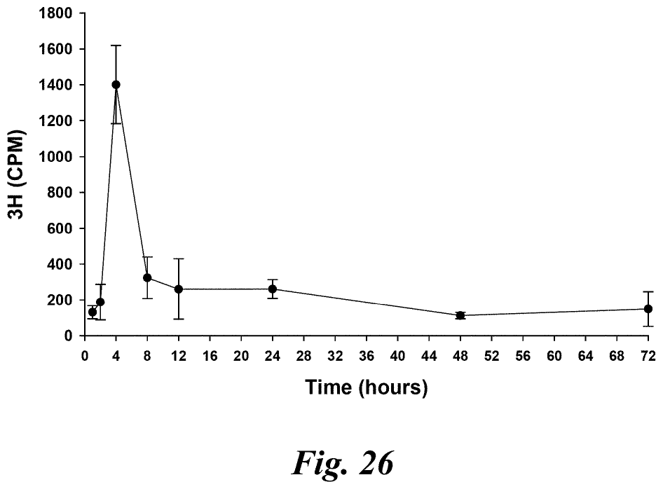

FIGS. 21-24 show intranasal Bryostatin-1 uptake into different organs over time. FIG. 25 shows the uptake and excretion of Bryostatin-1 following an intranasal dosing at 8-48 h. It was found that relatively high hippocampal uptake rates were achieved and maintained by this method with better retention compared to other “direct” contact organs, e.g. the lung (FIG. 25 and FIG. 26), which show a peak at 4 h and rapid excretion by 8 h. This series of studies shows that intranasal dosing may represent the optimal method for achieving sustained and high levels of Bryostatin-1 uptake for AD studies and human AD therapy.

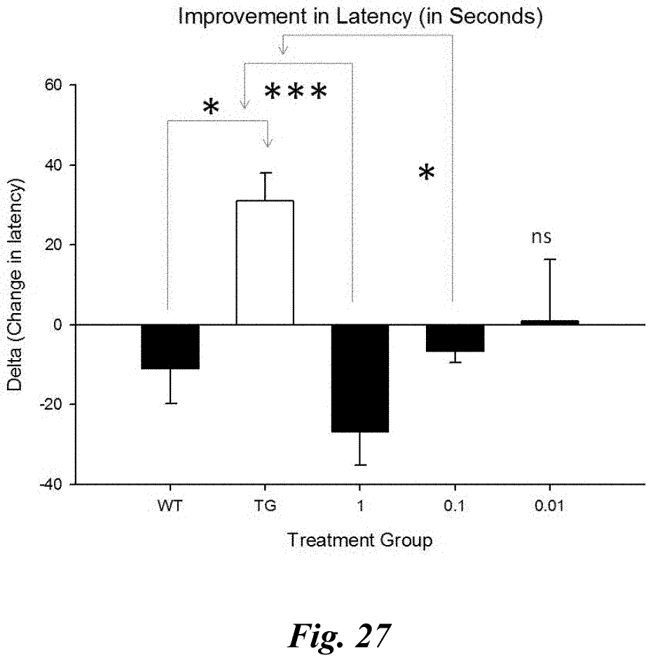

FIG. 27 shows Improvement in Latency. Bryostatin-1 significantly improves inter-trial latency in a mouse model of Down syndrome. Control differs from Down transgene (TG) (*). Latency improved with 1 μg, (***) and 0.1 μg (*) but not 0.01 μg. *P<0.05, ***P<0.001 vs. WT Error bars shown on SEM.

TS65DN mouse model of Down syndrome were purchased from MMRRC/Jackson Labs at 20-24 weeks of age and maintained in the LSUHSC-S vivarium. Segmentally trisomic Ts65Dn mice provide a postnatal model for Down syndrome. These mice were studied at 4 months of age when they exhibit significant cognitive impairment but retain good physical condition in the water maze. They were treated with either 1 μg, 0.1 or 0.01 μg of Bryostatin-1 in PET formulation (at a concentration of 33 μg/ml) by intranasal route on alternate days during week 1 and then daily for 5 days during water maze testing. Mice were briefly anesthetized using halothane system to administer Bryostatin-1 and allowed to recover for 2 h before testing in the Morris water maze.

Intranasal administration has the additional benefit of two routes of administration, nasal and oral (oral since a large fraction of unabsorbed drug is inadvertently swallowed after not being absorbed in the nose).

These are also the first data to show that intranasal Bryostatin-1 can affect performance in the TS65DN model. Radioactive uptake data have demonstrated high levels of Bryostatin-1 uptake into the hippocampus. This approach indicates that intranasal delivery of Bryostatin-1 may represent an important and novel treatment modality in human Down syndrome. We are still evaluating different behavioral aspects of this model to identify other behaviors which are significantly improved in this model by Bryostatin-1 treatment.

Therefore, we have described the present invention with respect to preferred embodiments with the understanding that these embodiments are capable of modification and alteration without departing from the teaching herein. Therefore, the present invention should not be limited to the precise details, but should encompass the subject matter of the claims that follow.

Thus, we have disclosed embodiments of the present invention based on our present understanding of the best mode to make and use these compounds. Those skilled in the art will readily understand that such preferred embodiments are subject to alteration and modification and therefore the present invention should not be limited to the precise details, but should encompass the subject matter of the claims that follow and their equivalents.

What is claimed is:

1. A method of treating neuro-degenerative disease comprising nasally administering to a patient in need thereof an effective amount of Bryostatin held in a plurality of microspheres, wherein said Bryostatin is selected from the group consisting of Bryostatin 1-20, wherein each of said microspheres comprises a polymer and the Bryostatin, and wherein said microspheres have a diameter of one to 1000 nanometers, wherein the polymer consists of a poly(D,L-lactide-co-glycoside), and wherein the microspheres are held in a nasal dosage form selected from the group consisting of sprays, mists, powders and droplets.

2. The method of claim 1 wherein said poly(D,L-lactide-co-glycoside) has a ratio of lactide to glycolic acid to be 25-75% lactide with the remaining comprising glycolic acid.

3. The method of claim 1 wherein said microspheres are lyophilized for reconstitution in an aqueous solution.

4. The method of claim 1 wherein said effective amount of Bryostatin is approximately 0.1 μg to 10 μg.

5. The method of claim 1 wherein said effective amount of Bryostatin is approximately from 0.5 μg to 2.0 μg.

6. The method of claim 1, wherein the dosage form for nasal administration comprises at least one of an aerosol, a spray, or a mist for administration to lungs or nasal passageways.

7. The method of claim 1, wherein an effective amount of Bryostatin is administered nasally to a patient for the treatment of Hutchinson Disease.

8. The method of claim 1, wherein an effective amount of Bryostatin is administered nasally to a patient for the treatment of Alzheimer's Disease.

9. The method of claim 1, wherein an effective amount of Bryostatin is administered nasally to a patient for the treatment of Parkinson's Disease.

10. The method of claim 1, wherein an effective amount of Bryostatin is administered nasally to a patient for the treatment of Down Syndrome.

11. A method of manufacturing an effective amount of Bryostatin held in a plurality of microspheres, wherein said Bryostatin is selected from the group consisting of Bryostatin 1-20, wherein each of said microspheres comprises a polymer and the Bryostatin, and wherein said microspheres have a diameter of one to 1000 nanometers, wherein the polymer consists of a poly(D,L-lactide-co-glycoside), and wherein the microspheres are held in a nasal dosage form selected from the group consisting of sprays, mists, powders and droplets for treating neuro-degenerative disease comprising nasally administering to a patient in need thereof.

12. The method of claim 11 wherein said poly(D,L-lactide-co-glycoside) has a ratio of lactide to glycolic acid to be 25-75% lactide with the remaining comprising glycolic acid.

13. The method of claim 11 wherein said microspheres are lyophilized for reconstitution in an aqueous solution.

14. The method of claim 11 wherein said effective amount of Bryostatin is approximately 0.1 μg to 10 μg.

15. The method of claim 11 wherein said effective amount of Bryostatin is approximately from 0.5 μg to 2.0 μg.

16. An article of manufacture comprising an effective amount of Bryostatin held in a plurality of microspheres, wherein said Bryostatin is selected from the group consisting of Bryostatin 1-20, wherein each of said microspheres comprises a polymer and the Bryostatin, and wherein said microspheres have a diameter of one to 1000 nanometers, wherein the polymer consists of a poly(D,L-lactide-co-glycoside), and wherein the microspheres are held in a nasal dosage form selected from the group consisting of sprays, mists, powders and droplets for treating neuro-degenerative disease comprising nasally administering to a patient in need thereof.

17. The article of manufacture of claim 16, wherein an effective amount of Bryostatin is administered nasally to a patient for the treatment of Hutchinson Disease.

18. The article of manufacture of claim 16, wherein an effective amount of Bryostatin is administered nasally to a patient for the treatment of Alzheimer's Disease.

19. The article of manufacture of claim 16, wherein an effective amount of Bryostatin is administered nasally to a patient for the treatment of Parkinson's Disease.

20. The article of manufacture of claim 16, wherein an effective amount of Bryostatin is administered nasally to a patient for the treatment of Down Syndrome.
